(19) United States
(12) Patent Application Publication
(10) Pub. No.: US 2005/0202453 A1
(43) Pub. Date: Sep. 15, 2005

(54) Nucleic acid amplification in yeast

(76) Inventors: Wen-Yuan Song, Gainesville, FL (US); Xiaodong Ding, Gainesville, FL (US); William Farmerie, Gainesville, FL (US)

Correspondence Address: AKERMAN SENTERFITT, P.O. BOX 3188, WEST PALM BEACH, FL, 33402-3188, US

(21) Appl. No.: 10/856,063

(22) Filed: May 28, 2004

Related U.S. Application Data
(60) Provisional application No. 60/474,090, filed on May 28, 2003.

(51) Int. Cl. C12Q 1/68; C12P 19/34; C12N 1/18; C12N 15/74

(52) U.S. Cl. 435/6; 435/91.2; 435/483; 435/254.2

(57) ABSTRACT

Plasmid DNA from single yeast colonies was efficiently amplified using rolling circle amplification (RCA). The amplified DNA was directly used for restriction digestion, DNA sequencing, and yeast transformation. The RCA of plasmid DNA from single yeast colonies for direct retransformation of yeast simplifies conventional procedures for yeast two-hybrid analysis and is suitable for high-throughput analyses. In summary, we have developed several methods to manipulate plasmid DNA in yeast. It will greatly simplify a number of yeast-based molecular biology tools, particularly yeast two-hybrid analysis. The methods are very useful for high-throughput assays.

### PRIORITY DATA

This application claims the benefit of provisional patent application U.S. Ser. No. 60/474,090 filed May 28, 2003 which is incorporated by reference, herein, in its entirety.

### STATEMENT AS TO FEDERALLY SPONSORED RESEARCH

The invention was made with U.S. government support under grant number DBI-0217312 awarded by the National Science Foundation. The U.S. government may have certain rights in the invention.

### FIELD OF THE INVENTION

The invention relates generally to the fields of microbiology, molecular biology and genetics. More particularly, the invention relates to a method of amplifying DNA from yeast cells.

### BACKGROUND

Yeast is a powerful tool for studying molecular biology in eukaryotes. The yeast one-hybrid, two-hybrid, and three-hybrid systems have been widely used to investigate protein-DNA and protein-protein interactions (Brent and Finley, 1997). In particular, the yeast two-hybrid system has recently evolved from a means to study interactions between small numbers of proteins to a tool that can be used to establish a protein-protein interaction database on a genome-wide scale (Uetz et al., 2000; Ito et al., 2001; Giot et al., 2003; Li et al., 2004). However, yeast is not ideal for direct analysis of plasmid DNA. Typically, only a small quantity of plasmid DNA can be isolated from yeast cells using conventional procedures. The recovered plasmids are not sufficient for many routine molecular assays such as restriction digestion and DNA sequencing. In addition, a disadvantage of yeast two-hybrid analysis is a significant number of false positives. To reduce them, it is important to show that the identified interactions are reproducible by re-transforming yeast with the constructs encoding the BD (bait) and AD (prey) fusions. As a negative control, the prey is often tested for no interactions with unrelated baits, including the vector encoding empty BD. These experiments are labor-intensive and time-consuming largely because the plasmid DNA isolated from yeast cultures is not sufficient for subsequent yeast retransformation. Therefore, further propagation of the recovered DNA in bacterial hosts becomes essential for conventional yeast two-hybrid analysis. However, this strategy is an impediment to high-throughput analyses.

There is therefore, a need in the art to provide high-throughput assays that are reproducible, low cost and lack errors.

### SUMMARY

The invention relates to methods and compositions for amplifying DNA from a yeast cell or isolated plasmids from yeast using rolling circle amplification (RCA). Using methods described herein, plasmid DNA from single yeast colonies was efficiently amplified by a phage DNA polymerase Φ29 DNA polymerase). The amplified DNA was directly used for restriction digestion, DNA sequencing, and yeast transformation. The high-fidelity and processivity of the Φ₂₉ DNA polymerase make it particularly suited for whole-plasmid amplification, transformation and use in functional screening assays. The RCA of plasmid DNA from single yeast colonies for direct retransformation of yeast significantly simplifies the conventional procedures for a yeast two-hybrid analysis and is particularly suitable for high-throughput analyses which allow comprehensive studies of protein-protein interactions in complex genomes.

In a preferred embodiment, the invention provides a method for amplifying nucleic acid molecules from a small amount of cells or template. The method comprises the steps of: providing isolated cells; administering a vector to the cells; placing the cell in a reaction mixture comprising a DNA polymerase and at least one primer under reaction conditions that allow amplification of the nucleic acid molecule. Preferably the cell is a eukaryotic cell, more preferably, the cell is a yeast cell. the vector preferably comprises a selectable marker.

In accordance with the invention, the yeast cell is treated with an enzyme, preferably with zymolase, as described in detail in the Examples which follow.

In another preferred embodiment, the DNA polymerase is selected from the group consisting of Φ29, Φ15, PZA, PZE, BS32, B103, Nf, M2, ΦPRD1, exo(−)VENT™, Klenow fragment of DNA polymerase I, T5. DNA polymerase, Sequenase, PRD 1 DNA polymerase, and T4 DNA polymerase. Preferably, the polymerase is a phage DNA polymerase, the preferred polymerase is Φ29 DNA polymerase.

In another preferred embodiment, the gene of interest or nucleic acid molecule is amplified by rolling circle amplification. In one aspect of the invention the nucleic acid molecule is circular DNA and can be in the range of between about 100 bases long up to several hundred kilobases long. Preferably up to about, 100 000 bases long, more preferably, up to about 200 000 bases long, more preferably, up to about 300 000, 400 000, and 500 000 bases long.

In another preferred embodiment, RCA method of the invention efficiently and faithfully amplifies circular plasmid DNA from yeast in a high fidelity manner. That is, the error rate for this method is between about 10⁻⁶-10⁻¹⁰.

In another preferred embodiment, the amplified DNA is digested and sequenced without further purification and preferably the high fidelity amplified DNA is transformed into yeast.

In another preferred embodiment, the RCA amplified DNA is concatemeric and preferably transforms eukaryotic cells, such as for example, yeast cell. A preferred method is use of a two-hybrid yeast analysis comprising bait and prey. For example, a bait vector, pDBLeu carries the yeast gene CYH2 that makes the yeast strain MaV203 (MAT˜, leu2-3, 112 trp1-901, his3˜200, ade2-101, gal4˜, gal80˜, SPAL10::URA3, GAL1::lacZ, HIS3_{UAS}_{^{GAL1}}::HIS3@LYS2, can1^{R}, cyh2^{R}). The RCA-amplified bait is, for example, pPC97 vector or pPC97—XA21KT. The prey is the sequence of interest which is detected by the bait.

In another preferred embodiment, the RCA method co-amplifies both bait and prey from yeast. Preferably, the interactions identified by yeast two-hybrid analysis, are specific, as it is preferred that the original bait is removed from the amplified products.

In another preferred embodiment, the original bait is removed from the amplified products, providing specificity of the amplified nucleic acid molecules, i.e., no further purification is necessary to remove the original bait from the amplified nucleic acid molecules (prey). A preferred method for removing the original bait from the amplified products is by counterselection, preferably using cycloheximide for counterselection.

In another preferred embodiment, the primers used to amplify the gene of interest or nucleic acid molecule comprise random primers that hybridize to the nucleic acid molecule in the reaction mixture. The primers of the invention are identified by any one of SEQ ID NO's 1 through 8. Preferably, one or any combination of primers identified by SEQ ID NO's 1 through 8 are administered to the reaction mixture.

In another preferred embodiment, primers with at least about 45% homology to the primers identified by any one of SEQ ID NO's 1-8 amplify nucleic acid molecules can be used, more preferably, primers with at least about 50% homology to the primers identified by any one of SEQ ID NO's 1-8 amplify nucleic acid molecules can be used, more preferably, primers with at least about 75% homology to the primers identified by any one of SEQ ID NO's 1-8 amplify nucleic acid molecules can be used, more preferably with at least about 80% homology to the primers identified by any one of SEQ ID NO's 1-8 amplify nucleic acid molecules can be used, more preferably, primers with at least about 95% homology to the primers identified by any one of SEQ ID NO's 1-8 amplify nucleic acid molecules can be used.

In another preferred embodiment, the primers comprise at least one modified base, preferably, the primers comprise about two modified bases, preferably the primers comprise up to 8 modified bases.

In a preferred embodiment, the vector comprises a selectable marker which can be an auxotrophic or antibiotic resistance marker.

In another preferred embodiment, a method for identifying a compound that interacts with amplified gene isolated from a mammal is provided. The method comprises contacting a candidate agent with the an amplified gene, an allele or fragment thereof, or expression product thereof; and, performing a detection step to detect interaction between the gene, an allele or fragment thereof, or expression product thereof.

Preferably, wherein the candidate compound is selected from the group consisting of a protein, a peptide, an oligopeptide, a nucleic acid, a small organic molecule, a polysaccharide and a polynucleotide.

In one aspect of the invention, the gene, variants or fragments thereof, or oligopeptides or candidate compound comprises a label.

In a preferred embodiment, the gene is isolated from a mammal suffering from or susceptible to a disease. The disease can be hereditary, a tumor, or caused by an infectious agent. The infectious agent is a virus, bacterium, protozoan or fungus.

In one preferred embodiment, the amplified gene, variant or fragment oligopeptide are provided on a solid support and binding of the candidate compound with the amplified gene, variant or fragment or oligopeptide is detected using a detectable label or read-out marker. Identification of a compound that interacts with the amplified nucleic acid molecule is a potential drug compound for therapeutic uses.

In another preferred embodiment, the invention provides a kit comprising an isolated yeast cell; zymolase; a vector; a reaction mixture comprising a DNA polymerase; primers identified by any one of SEQ ID NO's 1-8. The kit optionally provides instructions for carrying out the method are provided.

In another preferred embodiment, a method for identifying a component of a test sample, comprising: contacting a test sample with an amplified gene, variant or fragment thereof, or expression product of the amplified gene, variant or fragment thereof; and detecting interaction of the test sample with the amplified gene, an variant or fragment thereof, or expression product of the amplified gene, variant or fragment thereof. The test sample is a mammalian tissue or fluid sample.

In another preferred embodiment, the invention provides a method for identifying one or more genes that mediate susceptibility to disease susceptibility in a mammal comprising: amplifying nucleic acid molecules from a mammal, hybridizing an isolated nucleic acid sequence with a nucleic acid probe to form a hybridized molecule; and detecting sequences hybridized to the probe. The amplified gene, allele or fragment oligopeptide are provided on a solid support and binding of the candidate gene and/or gene product with the amplified gene, allele or fragment or oligopeptide is detected.

In accordance with the invention, the amplified gene sequence is compared to known genes and identified in a database. The database is GenBank, Human genome project or EMBL.

Although methods and materials similar or equivalent to those described herein can be used in the practice or testing of the present invention, suitable methods and materials are described below. All publications, patent applications, patents and other references mentioned herein are incorporated by reference in their entirety. In the case of conflict, the present specification, including definitions will control. The particular embodiments discussed below are illustrative only and not intended to be limiting.

Other aspects of the invention are described infra.

### BRIEF DESCRIPTION OF THE DRAWINGS

FIG. 1 is an agarose gel of amplification products of the plasmids pBOK-XA21K and pPC86-UK from yeast cells using RCA.

FIG. 2A through 2D is a series of agarose gels of amplification products resulting from optimization of RCA using yeast cells as template. (A) 3 day-old yeast cells pretreated by freeze/thaw and zymolase digestion. Control, no pretreatment. (B) Time course of RCA was performed with 3-day old cells and zymolase digestion. (C) Yeast cells were cultured for different time periods on medium and pretreated with zymolase. (D) Glycerol stocks with different dilution were used as templates. The cells were treated with zymolase before amplification.

FIG. 3 is an electropherogram of sequence data from RCA-amplified products.

FIG. 4. shows the results from β-galactosidase assays of RCA transformed yeast cells counterselected by cycloheximide. The RCA amplified pDBLeu-XA21KT and pPC86-UK were transformed into yeast containing either pPC97—XA21KT or the empty vector pPC97. The transformants grown on selective medium with or without cycloheximide were subjected to β-galactosidase assays. Each spot represents cells pooled from approximately 1000 individual colonies. *RCA products: pDBLeu-XA21KT and pPC86-UK.

FIG. 5. shows the results of interactors from a counter-selection assay. The verification of interactors using cycloheximide counterselection in a 96-well format. Yeast cells, containing the constructs indicated on the right, were transformed with the RCA amplified bait and prey and grown on the selective medium specified on the left. Colonies capable of growing on selective medium (SD/-Leu-Trp+Cycloheximide) were replicated on the SD/-Leu-Trp-His+Cycloheximide medium. The arrow indicates a colony growing poorly on selective medium, representing a false positive.

FIGS. 6A and 6B are agarose gels showing the results of RCA amplification and enzyme digested products. FIG. 6A shows the amplification of the plasmid pCmPU-GUS from agrobacterial cells using rolling circle amplification. Single bacterial colonies were picked and amplified in 10 μl volumes as described in the text. One microliter of the amplified DNA was digested overnight at 37° C. with the restriction enzyme SpeI. Restriction digestion of the pCmPU-GUS plasmid purified from E. coli is also shown as the control. The samples were electrophoretically separated in an agarose gel. FIG. 6B is an agarose gel electrophoresis of rescued plasmids from the E. coli strain DY329 transformed with rolling circle amplification (RCA) products. Bacterial cells were transformed with the RCA amplified pCmPU-GUS DNA using standard electroporation procedures. Plasmids were recovered from 10 independent transformants and analyzed by agarose gel electrophoresis. The control (pCmPU-GUS), uncut (upper) and SpeI digested (lower) samples are shown.

FIG. 7 shows the results from yeast two-hybrid assays using RCA amplified products. Yeast cells transformed with the indicated constructs were grown on the selective medium specified on the right. Colonies capable of growing on the SD medium without leucine and tryptophan show the presence of the transformed plasmids, whereas colonies growing on the SD medium lacking leucine, tryptophan and histidine indicate activation of the reporter gene His3 resulting from the interactions of the XA21K and UK fusion proteins in the yeast cells. Left panel (control), the plasmid DNA used for transformation was isolated from E. coli using conventional methods; right panel (RCA), the DNA was amplified from the yeast cells carrying the indicated plasmids.

FIG. 8 shows the results of an agarose gel electrophoresis of the plasmids rescued from the yeast cells transformed with RCA products. Yeast cells were transformed with the RCA amplified pPC86-UK DNA. Plasmids were recovered from 10 independent transformants, propagated in E. coli, and analyzed by agarose gel electrophoresis. Both uncut (upper) and SalI-NotI digested (lower) samples are shown. Control, pPC86-UK.

FIG. 9 shows the results from a high-throughput amplification of the bait pDBLeu-XA21KT and prey from the yeast cells using RCA. Cell lysates from single yeast colonies were used as templates for the amplification. The amplified DNA was digested with SalI and NotI. The samples were resolved by agarose gel electrophoresis.

### DETAILED DESCRIPTION

The invention provides methods and compositions for amplifying DNA from yeast cells using RCA. In one example of these methods, plasmid DNA is amplified from single yeast colonies using RCA. The amplified DNA is sufficient for restriction analysis, DNA sequencing and retransformation of yeast cell for confirmation of the initial yeast two-hybrid interactions.

Before the present invention is disclosed and described, it is to be understood that this invention is not limited to the particular structures, process steps, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

Definitions

In accordance with the present invention and as used herein, the following terms are defined with the following meanings, unless explicitly stated otherwise.

As used herein, “a”, “an,” and “the” include plural references unless the context clearly dictates otherwise.

The term “biomolecule” refers to DNA, RNA (including mRNA, rRNA, tRNA and tmRNA), nucleotides and nucleosides.

A base “position” as used herein refers to the location of a given base or nucleotide residue within a nucleic acid.

A “nucleic acid of interest,” as used herein, is any particular nucleic acid one desires to study in a sample.

The term “nucleic acid” may refer either to a molecule of DNA of indeterminate length or to a molecule of RNA of indeterminate length. In some aspects of the invention biomolecules and/or nucleic acids may be produced using a variety of known techniques, however, the preferred technique is rolling-circle amplification (RCA). Other techniques may include, for example, polymerase chain reaction (PCR) amplification, reverse-transcriptase polymerase chain reaction (RT-PCR) amplification, oligo ligation amplification (OLA), or single nucleotide primer extension reaction (SNuPE). Such techniques are well known to one skilled in the art and further are described in laboratory manuals such as Sambrook et al, (“Molecular Cloning: A Laboratory Manual”, Third edition, Cold Spring Harbor Laboratory, 2001) or Ausubel et al. (“Current Protocols in Molecular Biology”, John Wiley & Sons, 1998) both of which are incorporated herein by reference in their entirety, including any drawings, figures or tables.

As used herein, the term “nucleic acid primer” or “nucleic acid probe” are used interchangeably and refers to an oligonucleotide or polynucleotide that is capable of hybridizing to another nucleic acid of interest. A nucleic acid probe may occur naturally as in a purified restriction digest or be produced synthetically, recombinantly or by PCR amplification. As used herein, the term “nucleic acid probe”, refers to the oligonucleotide or polynucleotide used in a method of the present invention. That same oligonucleotide could also be used, for example, in a PCR method as a primer for polymerization, but as used herein, that oligonucleotide would then be referred to as a “primer”. Herein, oligonucleotides or polynucleotides may contain some modified linkages such as a phosphorothioate bond.

“Amplification” relates to the production of additional copies of a nucleic acid sequence. As used herein, amplification of a nucleic acid sequence is carried out by the Rolling Circle Amplification (RCA) method. Amplification is generally carried out using polymerase chain reaction (PCR) technologies or by isothermal amplification technologies well known in the art.

In either a linear or circular DNA molecule, discrete elements are referred to as being “upstream” or “5′” relative to an element if they are bonded or would be bonded to the 5′-end of that element. Similarly, discrete elements are “downstream” or “3′” relative to an element if they are or would be bonded to the 3′-end of that element.

Transcription proceeds in a 5′ to 3′ manner along the DNA strand. This means that RNA is made by the sequential addition of ribonucleotide-5′-triphosphates to the 3′-terminus of the growing chain (with the elimination of pyrophosphate).

As used herein, the term “target nucleic acid” or “nucleic acid target” refers to a particular nucleic acid sequence of interest. Thus, the “target” can exist in the presence of other nucleic acid molecules or within a larger nucleic acid molecule.

As used herein, “molecule” is used generically to encompass any vector, antibody, protein, drug and the like which are used in therapy and can be detected in a patient by the methods of the invention. For example, multiple different types of nucleic acid delivery vectors encoding different types of genes which may act together to promote a therapeutic effect, or to increase the efficacy or selectivity of gene transfer and/or gene expression in a cell. The nucleic acid delivery vector may be provided as naked nucleic acids or in a delivery vehicle associated with one or more molecules for facilitating entry of a nucleic acid into a cell. Suitable delivery vehicles include, but are not limited to: liposomal formulations, polypeptides; polysaccharides; lipopolysaccharides, viral formulations (e.g., including viruses, viral particles, artificial viral envelopes and the like), cell delivery vehicles, and the like.

As used herein, the term “administering a molecule to a cell” (e.g., an expression vector, nucleic acid, a delivery vehicle, agent, and the like) refers to transducing, transfecting, microinjecting, electroporating, or shooting, the cell with the molecule. In some aspects, molecules are introduced into a target cell by contacting the target cell with a delivery cell (e.g., by cell fusion or by lysing the delivery cell when it is in proximity to the target cell).

The term “polymorphism” refers to the coexistence of more than one form of a gene or portion (e.g., allelic variant) thereof. A portion of a gene of which there are at least two different forms, i.e., two different nucleotide sequences, is referred to as a “polymorphic region of a gene”. A specific genetic sequence at a polymorphic region of a gene is an allele. A polymorphic region can be a single nucleotide, the identity of which differs in different alleles. A polymorphic region can also be several nucleotides long.

As used herein, the term “specifically hybridizes” or “specifically detects” refers to the ability of a nucleic acid molecule to hybridize to at least approximately 6 consecutive nucleotides of a sample nucleic acid.

As used herein, the term “stringency” is used in reference to the conditions of temperature, ionic strength, and the presence of other compounds, under which nucleic acid hybridizations are conducted. With “high stringency” conditions, nucleic acid base pairing will occur only between nucleic acid fragments that have a high frequency of complementary base sequences. Thus, conditions of “weak” or “low” stringency are often required when it is desired that nucleic acids which are not completely complementary to one another be hybridized or annealed together. The art knows well that numerous equivalent conditions can be employed to comprise low stringency conditions.

As used herein, the term “Tm” is used in reference to the “melting temperature”. The melting temperature is the temperature at which 50% of a population of double-stranded nucleic acid molecules becomes dissociated into single strands. The equation for calculating the Tm of nucleic acids is well-known in the art. The Tm of a hybrid nucleic acid is often estimated using a formula adopted from hybridization assays in 1 M salt, and commonly used for calculating Tm for PCR primers: Tm=[(number of A+T)×2° C.+(number of G+C)×4° C.]. C. R. Newton et al. PCR, 2nd Ed., Springer-Verlag (New York: 1997), p. 24. This formula was found to be inaccurate for primers longer that 20 nucleotides. Other more sophisticated computations exist in the art which take structural as well as sequence characteristics into account for the calculation of Tm. A calculated Tm is merely an estimate; the optimum temperature is commonly determined empirically.

“Transcriptional regulatory sequence” is a generic term used throughout the specification to refer to DNA sequences, such as initiation signals, enhancers, promoters, silencing elements, which induce, inhibit or control transcription of protein coding sequences with which they are operably linked.

The term “vector” refers to a nucleic acid molecule, which is capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

A vector is a composition which can transduce, transfect, transform or infect a cell, thereby causing the cell to express nucleic acids and/or proteins other than those native to the cell, or in a manner not native to the cell. A cell is “transduced” by a nucleic acid when the nucleic acid is translocated into the cell from the extracelltilar environment. Any method of transferring a nucleic acid into the cell may be used; the term, unless otherwise indicated, does not imply any particular method of delivering a nucleic acid into a cell. A cell is “transformed” by a nucleic acid when the nucleic acid is transduced into the cell and stably replicated. A vector includes a nucleic acid (ordinarily RNA or DNA) to be expressed by the cell. A vector optionally includes materials to aid in achieving entry of the nucleic acid into the cell, such as a viral particle, liposome, protein coating or the like. A “cell transduction vector” is a vector which encodes a nucleic acid capable of stable replication and expression in a cell once the nucleic acid is transduced into the cell.

As used herein, a “target cell” or “recipient cell” refers to an individual cell or cell which is desired to be, or has been, a recipient of exogenous nucleic acid molecules, polynucleotides and/or proteins. The term is also intended to include progeny of a single cell.

“Label molecules” are chemical or biochemical moieties used for labeling a polynucleotide, a polypeptide, or an antibody. They include, but are not limited to, radionuclides, enzymes, substrates, cofactors, inhibitors, fluorescent agents, chromogenic agents, chemiluminescent agents, magnetic particles, and the like. Reporter molecules specifically bind, establish the presence of, and allow quantification of a particular polynucleotide, polypeptide, or antibody.

“Sample” is used herein in its broadest sense. A sample suspected of containing a nucleic acid can comprise a cell, chromosomes isolated from a cell (e.g., a spread of metaphase chromosomes), genomic DNA, RNA, cDNA and the like. A sample comprising polynucleotides, polypeptides, peptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; and the like.

“Substantially purified” refers to nucleic acid molecules or proteins that are removed from their natural environment and are isolated or separated, and are at least about 60% free, preferably about 75% free, and most preferably about 90% free, from other components with which they are naturally associated.

The terms “nucleic acid molecule” or “polynucleotide” will be used interchangeably throughout the specification, unless otherwise specified. As used herein, “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogues thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

As used herein, the term “fragment or segment”, as applied to a nucleic acid sequence, gene or polypeptide, will ordinarily be at least about 5 contiguous nucleic acid bases (for nucleic acid sequence or gene) or amino acids (for polypeptides), typically at least about 10 contiguous nucleic acid bases or amino acids, more typically at least about 20 contiguous nucleic acid bases or amino acids, usually at least about 30 contiguous nucleic acid bases or amino acids, preferably at least about 40 contiguous nucleic acid bases or amino acids, more preferably at least about 50 contiguous nucleic acid bases or amino acids, and even more preferably at least about 60 to 80 or more contiguous nucleic acid bases or amino acids in length. “Overlapping fragments” as used herein, refer to contiguous nucleic acid or peptide fragments which begin at the amino terminal end of a nucleic acid or protein and end at the carboxy terminal end of the nucleic acid or protein. Each nucleic acid or peptide fragment has at least about one contiguous nucleic acid or amino acid position in common with the next nucleic acid or peptide fragment, more preferably at least about three contiguous nucleic acid bases or amino acid positions in common, most preferably at least about ten contiguous nucleic acid bases amino acid positions in common.

A significant “fragment” in a nucleic acid context is a contiguous segment of at least about 17 nucleotides, generally at least 20 nucleotides, more generally at least 23 nucleotides, ordinarily at least 26 nucleotides, more ordinarily at least 29 nucleotides, often at least 32 nucleotides, more often at least 35 nucleotides, typically at least 38 nucleotides, more typically at least 41 nucleotides, usually at least 44 nucleotides, more usually at least 47 nucleotides, preferably at least 50 nucleotides, more preferably at least 53 nucleotides, and in particularly preferred embodiments will be at least 56 or more nucleotides.

Homologous nucleic acid sequences, when compared, exhibit significant sequence identity or similarity. The standards for homology in nucleic acids are either measures for homology generally used in the art by sequence comparison or based upon hybridization conditions. The hybridization conditions are described in greater detail below.

As used herein, “substantial homology” in the nucleic acid sequence comparison context means either that the segments, or their complementary strands, when compared, are identical when optimally aligned, with appropriate nucleotide insertions or deletions, in at least about 50% of the nucleotides, generally at least 56%, more generally at least 59%, ordinarily at least 62%, more ordinarily at least 65%, often at least 68%, more often at least 71%, typically at least 74%, more typically at least 77%, usually at least 80%, more usually at least about 85%, preferably at least about 90%, more preferably at least about 95 to 98% or more, and in particular embodiments, as high at about 99% or more of the nucleotides. Alternatively, substantial homology exists when the segments will hybridize under selective hybridization conditions, to a strand, or its complement, typically using a fragment derived from a known molecule. Typically, selective hybridization will occur when there is at least about 55% homology over a stretch of at least about 14 nucleotides, preferably at least about 65%, more preferably at least about 75%, and most preferably at least about 90%. See Kanehisa (1984) Nuc. Acids Res. 12:203-213. The length of homology comparison, as described, may be over longer stretches, and in certain embodiments will be over a stretch of at least about 17 nucleotides, usually at least about 20 nucleotides, more usually at least about 24 nucleotides, typically at least about 28 nucleotides, more typically at least about 40 nucleotides, preferably at least about 50 nucleotides, and more preferably at least about 75 to 100 or more nucleotides. The endpoints of the segments may be at many different pair combinations.

As used herein, the terms “complementary” or “complementarity” are used in reference to nucleic acids (i.e., a sequence of nucleotides) related by the well-known base-pairing rules that A pairs with T and C pairs with G. For example, the sequence 5′-A-G-T-3′, is complementary to the sequence 3′-T-C-A-5′. Complementarity can be “partial,” in which only some of the nucleic acid bases are matched according to the base pairing rules. On the other hand, there may be “complete” or “total” complementarity between the nucleic acid strands when all of the bases are matched according to base pairing rules. The degree of complementarity between nucleic acid strands has significant effects on the efficiency and strength of hybridization between nucleic acid strands as known well in the art. This is of particular importance in detection methods that depend upon binding between nucleic acids, such as those of the invention. The term “substantially complementary” refers to any probe that can hybridize to either or both strands of the target nucleic acid sequence under conditions of low stringency as described below or, preferably, in polymerase reaction buffer (Promega, M195A) heated to 95° C. and then cooled to room temperature. As used herein, when the nucleic acid probe is referred to as partially or totally complementary to the target nucleic acid, that refers to the 3′-terminal region of the probe (i.e. within about 10 nucleotides of the 3′-terminal nucleotide position).

“Substrate” refers to any rigid or semi-rigid support to which nucleic acid molecules or proteins are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

As used herein, “cancer” refers to all types of cancer or neoplasm or malignant tumors found in mammals, including, but not limited to: leukemias, lymphomas, melanomas, carcinomas and sarcomas. Examples of cancers are cancer of the brain, breast, pancreas, cervix, colon, head & neck, kidney, lung, non-small cell lung, melanoma, mesothelioma, ovary, sarcoma, stomach, uterus and Medulloblastoma.

Additional genes from cancers which can be identified by the disclosed method according to the invention include, for example, genes isolated from Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, and prostate cancer.

A “detectable marker gene” is a gene that allows cells carrying the gene to be specifically detected (e.g., distinguished from cells which do not carry the marker gene). A large variety of such marker genes are known in the art. Preferred examples thereof include detectable marker genes which encode proteins appearing on cellular surfaces, thereby facilitating simplified and rapid detection and/or cellular sorting. By way of illustration, the lacZ gene encoding beta-galactosidase can be used as a detectable marker, allowing cells transduced with a vector carrying the lacZ gene to be detected by staining.

A “selectable marker gene” is a gene that allows cells carrying the gene to be specifically selected for or against, in the presence of a corresponding selective agent. By way of illustration, an antibiotic resistance gene can be used as a positive selectable marker gene that allows a host cell to be positively selected for in the presence of the corresponding antibiotic. Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e. positive/negative) markers (see, e.g., WO 92/08796, published May 29, 1992, and WO 94/28143, published Dec. 8, 1994). Such marker genes can provide an added measure of control that can be advantageous in gene therapy contexts.

“Diagnostic” or “diagnosed” means identifying the presence or nature of a pathologic condition or a patient susceptible to a disease. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The terms “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

Rolling Circle Amplification (RCA)

RCA is an in vitro DNA amplification technique utilizing the rolling circle mechanism used by bacteria to replicate circular plasmids or viruses (Fire and Xu, 1995). The amplified DNA usually appears as tandemly repeated concatemers of the original template. By using random hexamer primers and Φ29 DNA polymerase, circular DNA templates can be amplified 10,000-fold at a constant temperature in a short time period (Dean et al., 2001). The Φ29 DNA polymerase has the capacity to perform displacement DNA synthesis for more than 70,000 base pairs without dissociation from the template and has high proofreading activity to ensure a high fidelity of amplification of DNA (Blanco et al., 1989; Garmendia et al., 1992). It has been reported that the error rate for this enzyme is 10⁻⁶-10⁻⁷ (Esteban et al., 1993).

In a preferred embodiment, maximum amplification of a desired nucleic acid molecule is preferred. Several parameters were tested to optimize the in vitro amplification of plasmid DNA from single yeast colonies using RCA. Preferably, maximum amplification is achieved using 3 day-old cells pretreated with zymolase as template. Preferably, the cells are yeast cells.

A detailed description of the amplification methods is described in the Examples which follow. For example, amplification from a small quantity of yeast glycerol stocks was also carried out, which allows further developments of high-throughput RCA. By using optimal conditions, 1-3 μg of plasmid DNA can be obtained from single colonies and yeast glycerol stocks within 12 hours. The amplified DNA is suitable for restriction digestion, DNA sequencing, yeast retransformation, and protein-protein interaction assays of encoded products.

In another preferred embodiment, the invention provides a RCA-based method to efficiently and faithfully, i.e., the error rate is low, amplify circular plasmid DNA from yeast in a high fidelity manner. That is, the error rate for this method is about 10⁻⁶-10^{-10.}

In another preferred embodiment, the amplified DNA is digested and sequenced without further purification and preferably the high fidelity amplified DNA is transformed into yeast.

In another preferred embodiment, the RCA amplified DNA is concatemeric and preferably transforms eukaryotic cells, such as for example, yeast cell. The RCA amplified DNA can also preferably transform, for example, E. coli strain DY329. This strain was engineered with a lambda prophage containing the recombination genes exo, bet, and gam under control of a temperature-sensitive lambda cI-repressor (Yu et al. 2000, Proc Natl Acad Sci USA. 97: 5978-5983.). Preferably, single units of the RCA-amplified plasmids can be recovered from the bacteria after transformation. This method is important for recovering RCA DNA from cells and is described in detail in the Examples which follow.

In another preferred embodiment, the RCA method disclosed herein, co-amplifies both bait and prey from yeast. To test specificity of interactions identified by yeast two-hybrid analysis, the original bait needs to be removed from the amplified products.

In another preferred embodiment, the original bait is removed from the amplified products. A preferred method for removing the original bait from the amplified products is by counterselection, preferably using cycloheximide for counterselection.

As an illustrative example, not meant to limit or construe the invention in any way, the following is provided. Among the methods developed to detect protein-protein interactions, yeast two-hybrid screening is simple, affordable, and sensitive. To reduce false positives, it is important to show that the identified interactions are reproducible by retransforming yeast with the constructs encoding the DNA binding (BD)¹ and activation fusions (also known as bait and prey, respectively). As a negative control, the prey is often tested for no interactions with unrelated baits, including the vector encoding empty BD. It is difficult, however, to include these steps in a large-scale analysis, mainly because the plasmid DNA isolated from yeast requires propagation in Escherichia coli to produce a sufficient amount of DNA for the subsequent experiments.

The example, herein, describes the co-amplification of bait and prey plasmids from yeast cells using rolling circle amplification (RCA) method as described in detail in the examples which follow. The bait construct is eliminated from the yeast transformants by using cycloheximide counterselection. The methods described infra, demonstrate the effectiveness and high-fidelity amplification of the RCA-based high-throughput system for characterization and verification of candidate interactors identified from initial yeast two-hybrid screening.

The RCA-amplified bait and prey can be directly transformed into yeast. To test for lack of interactions of the prey with unrelated baits, cycloheximide counterselection is used to exclude the original bait in the RCA products. The bait vector, for example, pDBLeu carries the yeast gene CYH2 that makes the yeast strain MaV203 (MAT˜, leu2-3, 112 trp1-901, his3˜200, ade2-101, gal4, gal80˜, SPAL10::URA3, GAL1::lacZ, HIS3_{UAS}_{^{GAL1}}::HIS3@LYS2, can1^{R}, cyh2^{R}) susceptible to the inhibitor. The RCA-amplified bait is, for example, pPC97 vector or pPC97-XA21KT. The two-hybrid vector pPC97 is similar to pDBLeu except for the lack of the CYH2 gene. Yeast transformants were plated on selective medium (SD/-Leu-Trp) with or without cycloheximide. Since XA21KT interacts with UK, the pPC97—XA21KT-containing yeast cells, transformed with the RCA-ampliWed pDBLeu-XA21KT and pPC86-50 UK, turned blue in the β-galactosidase assays regardless of the cycloheximide counterselection. When transformed with the RCA products, the cells carrying the pPC97 empty vector showed no detectable β-galactosidase activity in the presence of cycloheximide but a strong β-galactosidase activity in the absence of the inhibitor. Thus, cycloheximide counterselection, as is a preferred counterselector and can efficiently select against the presence of pDBLeu-XA21KT (bait) in the RCA products.

Compared with in vivo DNA propagation using E. coli, the RCA-based approach eliminates a number of laborious and time-consuming steps involved in the conventional yeast two-hybrid procedures, such as liquid culturing of yeast cells, plasmid isolation from the cultured yeast cells, transformation of E. coli using the rescued plasmid DNA, isolation of plasmids from the transformed E. coli for subsequent yeast transformation and DNA sequencing. Such operational steps are a major limitation for carrying out large-scale yeast two-hybrid screening.

In comparison to the PCR-mediated in vitro DNA amplification, the RCA system also has several advantages. It can efficiently amplify circular DNA ranging in size from small plasmids to bacterial artificial chromosomes with the size of several hundred thousand base pairs. The proofreading activity of Φ29 DNA polymerase (Garmendia et al. 1992; J. Biol. Chem. 267:2594-2599) ensures that RCA is a high-fidelity amplification this study). The random hexamer primers are universal for amplification of any plasmids, which eliminates the need for synthesis of gene-specific primers.

In another preferred embodiment, original circular plasmids rather than newly synthesized DNA as template for amplification are used as the error rate of amplification can be further reduced and the contamination risk is minimal. More importantly, the capability of high-fidelity amplification of large pieces of DNA comprising multiple genes enables complex functional assays requiring a cooperative action of multi-gene products. These features make the RCA products amenable to a yeast two-hybrid analysis, particularly on a large scale.

In accordance with the invention, the disclosed RCA-based method to manipulating plasmid DNA in yeast is reliable and robust. It is based on standard laboratory equipment and can be easily carried out in a high-throughput manner. This approach is applicable to other yeast-based systems such as the yeast one-hybrid, reverse yeast two-hybrid, and yeast three-hybrid screenings, thereby facilitating the utility of yeast as a tool for molecular biology and genomics research.

In an illustrative example, which is not meant to limit or construe the invention in any way, the following is provided:

A selected yeast stain carrying for example, two plasmid constructs is used to perform nucleic acid amplification using a rolling circle amplification method (RCA). The two plasmids are for example, shuttle plasmids and each can be distinguished by a variety of methods. One method can be antibiotic selection in a bacterial host cell, for example, E. coli. Each plasmid can confer resistance to a different antibiotic. For example, the first plasmid can confer kanamycin resistance, the second plasmid can confer, for example, ampicillin resistance. Another method for selection can be by complementation in yeast. For example, pBOK-XA21K: LEU2, pPC86-UK: TRP1 are described in the Examples which follow. Another method can be by their restriction patterns after digestion with restriction enzymes, for example SalI and NotI. To carry out amplification, preferably 3 day-old single colonies are picked directly from plates. The RCA reactions can be performed using the TempliPhi 100 Amplification kit (Amersham Biosciences, Piscataway, N.J.). For example, ⅓-½ of each single yeast colony can be picked using toothpicks and suspended into sample buffer (Amersham Biosciences, Piscataway, N.J.). For enzymatic treatment, zymolase (Zymo Research, Orange, Calif.) is added and the reaction mixtures incubated. For freeze/thaw treatment, the resuspended cells are frozen at −80° C. for example, and then thawed at room temperature. For amplification from glycerol stocks, yeast stocks are mixed with sample buffer. Amplified DNA is digested with restriction enzymes and the samples are resolved by agarose gel electrophoresis.

As discussed above, the readout system can be both growth on medium lacking an essential growth amino acid, such as for example, histidine and enzymatic activity of, for example, β-galactosidase which can be subsequently screened. Preferably, the combination of two components that constitute the readout system in many cases allows a more ready interpretation of results, in particular if one of the components, when activated, effects a change in color.

Preferably, the method is automated. For example, a colony picking robot is used to pick the resulting yeast colonies into individual wells of microtiter plates containing selective medium lacking an amino acid such as for example, histidine, leucine and the like, and the resulting plates are incubated at 30° C. to allow cell growth. The interaction library contained in microtiter plates can be optionally replicated and stored. Using a spotting robot, cells are transferred to replica membranes which are subsequently placed onto one each of the selective media(SD), for example, SD-leu-trp-his. After incubation on the selective plates, the clones grown on the membranes are subjected to a β-galactosidase assay and a digital image from each membrane is obtained with a CCD camera which is then stored on computer. Using digital image processing and analysis clones that express molecules of interest can be identified by considering the pattern of β-galactosidase activity from clones grown on the various selective media. Alternatively, restriction enzyme digests can also be used as described above. Following such methods, nucleic acid molecules are amplified using rolling circle amplification.

Two applications of interests are the application of a large scale two-hybrid system for the detection of protein-protein interactions involved in medically relevant pathways which may be useful as diagnostic or therapeutic targets for the treatment of disease, and a large scale tri-hybrid system for the identification of, for example, novel post-transciptional regulators and their binding sites.

In a further preferred embodiment, the vectors are plasmids, artificial chromosomes, viruses or other extrachromosomal elements.

Whereas it is preferred, due to the easy handling, to employ plasmids that specify the genetic elements in accordance with the present invention, the persons skilled in the art will be able to devise other systems that carry said genetic elements and that are identified above.

In an additional preferred embodiment, a readout system is a detectable protein. A number of readout systems are known in the art and may, if necessary, be adapted to be useful in the method of the invention. Such detection systems are described infra.

Most preferably, a detectable protein is one encoded, for example, by the genes for lacZ, HIS2, HIS3, LEU2, TRP, and the like. As is well known in the art, the expression of the β-galactosidase enzyme in yeast can be used for the formation of a detectable blue colony after incubation in β-Gal solution. Of course, the method of the invention is not restricted for use of only one readout system. On the contrary, if desired, a number of such readout systems may be combined. The combination of a number of readout systems is, in accordance with the present invention, also comprised by the term “readout system”.

Although the two-hybrid system has been developed in yeast, the method of the invention can be carried out in a variety of host systems. Preferred of those are yeast cells, bacterial cells, mammalian cells, insect cells or plant cells. Preferably, the bacterial cells are E. coli cells. Of course, the genetic elements may be engineered and prepared in one host organism and then, e.g. by employing shuttle vectors, be transferred to a different host organism where it is employed in the method of the invention.

The biological principal of counter-selection referred to above is well known in the art. Accordingly, the person skilled in the art may chose from a variety of such counterselectable markers. Preferably, said markers are kanamycin, cycloheximide, HIS2, HIS3, LEU2, TRP, and the like.

It is further preferred in accordance with the present invention that the selectable markers are auxotrophic or antibiotic markers.

It is important to note that some of the markers that are used as a readout system, may also be used as selectable markers. It is further important to note that one and the same marker can not be used as selectable marker and as part of the readout system at the same time.

In particular in cases where a large number of clones is to be analyzed, said transfer is advantageously effected or assisted by automation or a picking robot. Naturally, other automation or robot systems that reliably pick progeny of the host cells into predetermined arrays in the storage compartments may also be employed.

The host cells will, in this embodiment, be propagated in the storage compartment and provide further progeny for the additional tests. Preferably, replicas of the storage compartment maintaining the array of clones are set up. The storage compartments comprising the transformed host cells and the appropriate media may be maintained in accordance with conventional cultivation protocols. Alternatively, the storage compartments may comprise an anti-freeze agent and therefore be appropriate for storage in a deep-freezer. This embodiment is particularly useful when the evaluation of potential interacting partners is to be postponed. As is well known in the art, frozen host cells may easily be recovered upon thawing and further tested in accordance with the invention. Most preferably, the anti-freeze agent is glycerol which is preferably present in said media in an amount of 3-25% (vol/vol).

In a further particularly preferred embodiment of the method of the invention, the storage compartment is a microtiter plate. Most preferably, the microtiter plate comprises 96 or 384 wells. Microtiter plates have the particular advantage of providing a pre-fixed array that allows the easy replicating of clones and furthermore the unambiguous identification and assignment of clones throughout the various steps of the experiment. The 384 well microtiter plate is, due to its comparatively small size and large number of compartments, particularly suitable for experiments where large numbers of clones need to be screened.

Depending on the design of the experiment, the host cells may be grown in the storage compartment such as the above microtiter plate to logarithmic or stationary phase. Growth conditions may be established by the person skilled in the art according to conventional procedures. Cell growth is usually performed between 15 and 45 degrees Celsius.

The selective media used for growth of appropriate clones may be in liquid or in solid form. Preferably, the selective media when used in conjunction with a spotting robot and membranes as planar carriers are solidified with agar on which the spotted membranes are subsequently placed. Alternatively, and also preferably, the selective media when in liquid form are held within microtiter plates and the transfer is made by replication.

The readout system can be analyzed by a variety of means. For example, it can be analyzed by visual inspection, radioactive, chemiluminescent, fluorescent, photometric, spectrometric, infra red, calorimetric or resonant detection.

Most preferably, the identity of positive host cells and false positive host cells are stored on computer, for example within a relational database.

Primers

In a preferred embodiment, universal random hexamer primers are used for amplification of any plasmids. preferably, use of the disclosed random hexamer primers eliminates the need to synthesize gene-specific primers.

In a preferred embodiment, the random hexamer primers are identified by the following sequences as described in the Examples which follow.

(SEQ ID NO 1)

5′-AGGTCGACCCCGGGAATGAAAGGCCACCCATT-3′

(SEQ ID NO 2)

5′-GGGGTACCGGATCCTCAAAATTCAAGGCTCCCACCTTCAA-3′.

(SEQ ID NO 3)

5′-TCATCGGAAGAGAGTAG-3′

(SEQ ID NO 4)

5′-AGGGATGTTTAATACCACTAC-3′

(SEQ ID NO 5)

5′-TTGATTGGAGACTTGACC-3′,

(SEQ ID NO 6)

5′-CCCAACCAAGAAGTATGCTGA-3′

(SEQ ID NO 7)

5′-TCACAGGGAAGGAGACAATGGCA-3′

(SEQ ID NO 8)

5′-ATGAAATCATGTGTTGTTGTGGCA-3′

In another preferred embodiment, primers with at least about 45% homology to the primers identified by any one of SEQ ID NO's 1-8 are used to amplify nucleic acid molecules, preferably, primers with at least about 50% homology to the primers identified by any one of SEQ ID NO's 1-8 are used to amplify nucleic acid molecules, preferably, primers with at least about 75% homology to the primers identified by any one of SEQ ID NO's 1-8 are used to amplify nucleic acid molecules, preferably, primers with at least about 80% homology to the primers identified by any one of SEQ ID NO's 1-8 are used to amplify nucleic acid molecules, preferably, primers with at least about 95%, 96%, 97%, 98%, or 99.9% homology to the primers identified by any one of SEQ ID NO's 1-8 are used to amplify nucleic acid molecules.

In another preferred embodiment, any one or combination thereof, of primers identified by SEQ ID NO's 1-8 amplify circular nucleic acid molecules. Preferably, the nucleic acid molecules are DNA molecules. For example, plasmids, bacterial artificial chromosomes.

In another preferred embodiment, the nucleic acid molecules amplified by any one of SEQ ID NO's 1-8 are at least about 50 base pairs long, more preferably, the nucleic acid molecules amplified by any one of SEQ ID NO's 1-8 are at least about 100 base pairs long, more preferably, the nucleic acid molecules amplified by any one of SEQ ID NO's 1-8 are at least about 1000 base pairs long, more preferably, the nucleic acid molecules amplified by any one of SEQ ID NO's 1-8 are at least about 5000 base pairs long up to about 400 hundred kilo base pairs.

In accordance with the invention, any desired primer may be used. For example, primers for use in the disclosed amplification method can be oligonucleotides having sequence complementary to the target sequence. This sequence is referred to as the complementary portion of the primer. The complementary portion of a primer can be any length that supports specific and stable hybridization between the primer and the target sequence under the reaction conditions. Generally, for reactions at 37° C., this can be, for example about 5 to about 35 nucleotides long or about 16 to about 24 nucleotides long. If whole genome amplification is desired, the primers can be from about 5 to about 60 nucleotides long, and in particular, can be about 5, 6, 7, 8, 9, 10, 11, 12, 13, 14, 15, 16, 17, 18, 19, and/or 20 nucleotides long.

In a preferred embodiment, target sequences that are to be amplified are of unknown sequence. For example, nucleic acid isolated from a sample for which the sequence is from an individual or any organism. In such cases, primers may be random, for example, primers identified by SEQ ID NO's 1-8 or homologous sequences thereof, or of degenerate sequence (that is, use of a collection of primers having a variety of sequences), primer hybridization need not be specific. In such cases the primers need only be effective in priming synthesis. For example, in whole genome amplification specificity of priming is not essential since the goal generally is to amplify all sequences equally. Sets of random or degenerate primers can comprise primers of about 5, 6, 7, 8, 9, 10, 11, 12, 13, 14, 15, 16, 17, 18, 19, and/or 20 nucleotides long or more. Primers six nucleotides long are referred to as hexamer primers. For example, preferred primers for whole genome amplification are random hexamer primers. That is, random hexamer primers where every possible six nucleotide sequence is represented in the set of primers. Similarly, sets of random primers of other particular lengths, or of a mixture of lengths preferably comprise every possible sequence the length of the primer, or, in particular, the length of the complementary portion of the primer. Use of random primers is described in U.S. Pat. Nos. 5,043,272 and 6,214,587 the contents of which are hereby incorporated by reference in their entirety.

In another preferred embodiment, the disclosed primers, as described in the Examples which follow, can have one or more modified nucleotides. Such primers are referred to herein as modified primers. Modified primers have several advantages. First, some forms of modified primers, such as RNA/2′-O-methyl RNA chimeric primers, have a higher melting temperature (Tm) than DNA primers. This increases the stability of primer hybridization and will increase strand invasion by the primers. This will lead to more efficient priming. Also, since the primers are made of RNA, they will be exonuclease resistant. Such primers, if tagged with minor groove binders at their 5′ end, will also have better strand invasion of the template dsDNA. In addition, RNA primers can also be very useful for amplification of nucleic acid molecules from biological samples such as cells or tissue. Since the biological samples contain endogenous RNA, this RNA can be degraded with RNase to generate a pool of random oligomers, which can then be used to prime the polymerase for amplification of the DNA. This eliminates any need to add primers to the reaction. Alternatively, DNase digestion of biological samples can generate a pool of DNA oligonucleotide primers for RNA dependent DNA amplification.

Chimeric primers can also be used. Chimeric primers are primers having at least two types of nucleotides, such as both deoxyribonucleotides and ribonucleotides, ribonucleotides and modified nucleotides, or two different types of modified nucleotides. One form of chimeric primer is peptide nucleic acid/nucleic acid primers (PNA/NAP). For example, 5′-PNA-DNA-3′ or 5′-PNA-RNA-3′ primers may be used for more efficient strand invasion and polymerization invasion. The DNA and RNA portions of such primers can have random or degenerate sequences. Other forms of chimeric primers are, for example, 5′-(2′-O-Methyl)RNA-RNA-3′ or 5′-(2′-O-Methyl)RNA-DNA-3′.

Many modified nucleotides (nucleotide analogs) are known and can be used in oligonucleotides. A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl, hypoxanthin-9-yl (I), and 2-aminoadenin-9-yl. A modified base includes but is not limited to locked nucleic acids (LNA), 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and 0-6 substituted purines, including 2-aminopropyladenine, 5-propynyluracil and 5-propynylcytosine. 5-methylcytosine can increase the stability of duplex formation. Other modified bases are those that function as universal bases. Universal bases include 3-nitropyrrole and 5-nitroindole. Universal bases substitute for the normal bases but have no bias in base pairing. That is, universal bases can base pair with any other base. Primers composed, either in whole or in part, of nucleotides with universal bases are useful for reducing or eliminating amplification bias against repeated sequences in a target sample. This would be useful, for example, where a loss of sequence complexity in the amplified products is undesirable. Base modifications often can be combined with for example a sugar modification, such as 2′-O-methoxyethyl, to achieve unique properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference.

Nucleotide analogs can also include modifications of the sugar moiety. Modifications to the sugar moiety would include natural modifications of the ribose and deoxyribose as well as synthetic modifications. Sugar modifications include but are not limited to the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10, alkyl or C2 to C10 alkenyl and alkynyl. 2′ sugar modifications also include but are not limited to —O[(CH₂)ₙO]ₘ CH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m are from 1 to about 10.

Other modifications at the 2′ position include but are not limited to: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkages between two nucleotides can be through a 3′-5′ linkage or a 2′-5′ linkage, and the linkage can comprise inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

It is understood that nucleotide analogs need only comprise a single modification, but may also comprise multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize and hybridize to complementary nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. (See also Nielsen et al., Science 254:1497-1500 (1991)).

Primers can comprise nucleotides and can be made up of different types of nucleotides or the same type of nucleotides. For example, one or more of the nucleotides in a primer can be ribonucleotides, 2′-O-methyl ribonucleotides, or a mixture of ribonucleotides and 2′-O-methyl ribonucleotides; about 10% to about 50% of the nucleotides can be ribonucleotides, 2′-O-methyl ribonucleotides, or a mixture of ribonucleotides and 2′-O-methyl ribonucleotides; about 50% or more of the nucleotides can be ribonucleotides, 2′-O-methyl ribonucleotides, or a mixture of ribonucleotides and 2′-O-methyl ribonucleotides; or all of the nucleotides are ribonucleotides, 2′-O-methyl ribonucleotides, or a mixture of ribonucleotides and 2′-O-methyl ribonucleotides. The nucleotides can comprise bases (that is, the base portion of the nucleotide) and can (and normally will) comprise different types of bases. For example, one or more of the bases can be universal bases, such as 3-nitropyrrole or 5-nitroindole; about 10% to about 50% of the bases can be universal bases; about 50% or more of the bases can be universal bases; or all of the bases can be universal bases.

Primers may, but need not, also comprise additional sequence at the 5′ end of the primer that is not complementary to the target sequence. This sequence is referred to as the non-complementary portion of the primer. The non-complementary portion of the primer, if present, serves to facilitate strand displacement during DNA replication. The non-complementary portion of the primer can also include a functional sequence such as a promoter for an RNA polymerase. The non-complementary portion of a primer may be any length, but is generally about 1 to 100 nucleotides long, and preferably about 4 to 8 nucleotides long. The use of a non-complementary portion is not preferred when random or partially random primers are used for example, in whole genome amplification.

Detection Tags

The non-complementary portion of a primer can include sequences to be used to further manipulate or analyze amplified sequences. An example of such a sequence is a detection tag, which is a specific nucleotide sequence present in the non-complementary portion of a primer. Detection tags have sequences complementary to detection probes. Detection tags can be detected using their cognate detection probes. Detection tags become incorporated at the ends of amplified strands. The result is amplified DNA having detection tag sequences that are complementary to the complementary portion of detection probes. If present, there may be one, two, three, or more than three detection tags on a primer. It is preferred that a primer have one, two, three or four detection tags. Most preferably, a primer will have one detection tag. Generally, it is preferred that a primer have 10 detection tags or less. There is no fundamental limit to the number of detection tags that can be present on a primer except the size of the primer. When there are multiple detection tags, they may have the same sequence or they may have different sequences, with each different sequence complementary to a different detection probe. It is preferred that a primer comprise detection tags that have the same sequence such that they are all complementary to a single detection probe. For some multiplex detection methods, it is preferable that primers comprise up to six detection tags and that the detection tag portions have different sequences such that each of the detection tag portions is complementary to a different detection probe. A similar effect can be achieved by using a set of primers where each has a single different detection tag. The detection tags can each be any length that supports specific and stable hybridization between the detection tags and the detection probe. For this purpose, a length of about 10 to about 35 nucleotides is preferred, with a detection tag portion about 15 to about 20 nucleotides long being most preferred.

Address Tag

Another example of a sequence that can be included in the non-complementary portion of a primer is an address tag. An address tag has a sequence complementary to an address probe. Address tags become incorporated at the ends of amplified strands. The result is amplified DNA having address tag sequences that are complementary to the complementary portion of address probes. If present, there may be one, or more than one, address tag on a primer. It is preferred that a primer have one or two address tags. Most preferably, a primer will have one address tag. Generally, it is preferred that a primer have 10 address tags or less. There is no fundamental limit to the number of address tags that can be present on a primer except the size of the primer. When there are multiple address tags, they may have the same sequence or they may have different sequences, with each different sequence complementary to a different address probe. It is preferred that a primer comprise address tags that have the same sequence such that they are all complementary to a single address probe. The address tag portion can be any length that supports specific and stable hybridization between the address tag and the address probe. For this purpose, a length between about 10 and 35 nucleotides long is preferred, with an address tag portion between about 15 to 20 nucleotides long being most preferred.

Nucleic Acid Fingerprints

The disclosed method can be used to produce replicated strands that serve as a nucleic acid fingerprint of a complex sample of nucleic acid. Such a nucleic acid fingerprint can be compared with other, similarly prepared nucleic acid fingerprints of other nucleic acid samples to allow convenient detection of differences between the samples. The nucleic acid fingerprints can be used both for detection of related nucleic acid samples and comparison of nucleic acid samples. For example, the presence or identity of specific organisms can be detected by producing a nucleic acid fingerprint of the test organism and comparing the resulting nucleic acid fingerprint with reference nucleic acid fingerprints prepared from known organisms. Changes and differences in gene expression patterns can also be detected by preparing nucleic acid fingerprints of mRNA from different cell samples and comparing the nucleic acid fingerprints. The replicated strands can also be used to produce a set of probes or primers that is specific for the source of a nucleic acid sample. The replicated strands can also be used as a library of nucleic acid sequences present in a sample. Nucleic acid fingerprints can be made up of, or derived from, for example, whole genome amplification of a sample such that the entire relevant nucleic acid content of the sample is substantially represented, or from multiple strand displacement amplification of selected target sequences within a sample.

Nucleic acid fingerprints can be stored or archived for later use. For example, replicated strands produced in the disclosed method can be physically stored, either in solution, frozen, or attached or adhered to a solid-state substrate such as an array. Storage in an array is useful for providing an archived probe set derived from the nucleic acids in any sample of interest. As another example, informational content of, or derived from, nucleic acid fingerprints can also be stored. Such information can be stored, for example, in or as computer readable media. Examples of informational content of nucleic acid fingerprints include nucleic acid sequence information (complete or partial); differential nucleic acid sequence information such as sequences present in one sample but not another; hybridization patterns of replicated strands to, for example, nucleic acid arrays, sets, chips, or other replicated strands. Numerous other data that is or can be derived from nucleic acid fingerprints and replicated strands produced in the disclosed method can also be collected, used, saved, stored, and/or archived.

Nucleic acid fingerprints can also comprise or be made up of other information derived from the information generated in the disclosed method, and can be combined with information obtained or generated from any other source. The informational nature of nucleic acid fingerprints produced using the disclosed method lends itself to combination and/or analysis using known bioinformatics systems and methods.

Nucleic acid fingerprints of nucleic acid samples can be compared to a similar nucleic acid fingerprint derived from any other sample to detect similarities and differences in the samples (which is indicative of similarities and differences in the nucleic acids in the samples). For example, a nucleic acid fingerprint of a first nucleic acid sample can be compared to a nucleic acid fingerprint of a sample from the same type of organism as the first nucleic acid sample, a sample from the same type of tissue as the first nucleic acid sample, a sample from the same organism as the first nucleic acid sample, a sample obtained from the same source but at time different from that of the first nucleic acid sample, a sample from an organism different from that of the first nucleic acid sample, a sample from a type of tissue different from that of the first nucleic acid sample, a sample from a strain of organism different from that of the first nucleic acid sample, a sample from a species of organism different from that of the first nucleic acid sample, or a sample from a type of organism different from that of the first nucleic acid sample.

The same type of tissue is tissue of the same type such as liver tissue, muscle tissue, or skin (which may be from the same or a different organism or type of organism). The same organism refers to the same individual, animal, or cell. For example, two samples taken from a patient are from the same organism. The same source is similar but broader, referring to samples from, for example, the same organism, the same tissue from the same organism, the same DNA molecule, or the same DNA library. Samples from the same source that are to be compared can be collected at different times (thus allowing for potential changes over time to be detected). This is especially useful when the effect of a treatment or change in condition is to be assessed. Samples from the same source that have undergone different treatments can also be collected and compared using the disclosed method. A different organism refers to a different individual organism, such as a different patient, a different individual animal, different mono-cellular or multi-cellular organisms. Different organism includes a different organism of the same type or organisms of different types. A different type of organism refers to organisms of different types such as a dog and cat, a human and a mouse, or bacteria such as E. coli and Salmonella. A different type of tissue refers to tissues of different types such as liver and kidney, or skin and brain. A different strain or species of organism refers to organisms differing in their species or strain designation as those terms are understood in the art.

Solid-State Detectors

Solid-state detectors are solid-state substrates or supports to which address probes or detection molecules have been coupled. A preferred form of solid-state detector is an array detector. An array detector is a solid-state detector to which multiple different address probes or detection molecules have been coupled in an array, grid, or other organized pattern.

Solid-state substrates for use in solid-state detectors can include any solid material to which oligonucleotides can be coupled. This includes materials such as acrylamide, cellulose, nitrocellulose, glass, gold, polystyrene, polyethylene vinyl acetate, polypropylene, polymethacrylate, polyethylene, polyethylene oxide, glass, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids. Solid-state substrates can have any useful form including thin films or membranes, beads, bottles, dishes, fibers, optical fibers, woven fibers, chips, compact disks, shaped polymers, particles and microparticles. A chip is a rectangular or square small piece of material. Preferred forms for solid-state substrates are thin films, beads, or chips.

Address probes immobilized on a solid-state substrate allow capture of the products of the disclosed amplification method on a solid-state detector. Such capture provides a convenient means of washing away reaction components that might interfere with subsequent detection steps. By attaching different address probes to different regions of a solid-state detector, different amplification products can be captured at different, and therefore diagnostic, locations on the solid-state detector. For example, in a multiplex assay, address probes specific for numerous different amplified nucleic acids (each representing a different target sequence amplified via a different set of primers) can be immobilized in an array, each in a different location. Capture and detection will occur only at those array locations corresponding to amplified nucleic acids for which the corresponding target sequences were present in a sample.

Methods for immobilization of oligonucleotides to solid-state substrates are well established. Oligonucleotides, including address probes and detection probes, can be coupled to substrates using established coupling methods. For example, suitable attachment methods are described by Pease et al., Proc. Natl. Acad. Sci. USA 91(11):5022-5026 (1994), and Khrapko et al., Mol. Biol. (Mosk) (USSR) 25:718-730 (1991). A method for immobilization of 3′-amine oligonucleotides on casein-coated slides is described by Stimpson et al., Proc. Natl. Acad. Sci. USA 92:6379-6383 (1995). A preferred method of attaching oligonucleotides to solid-state substrates is described by Guo et al., Nucleic Acids Res. 22:5456-5465 (1994). Examples of nucleic acid chips and arrays, including methods of making and using such chips and arrays, are described in U.S. Pat. Nos. 6,287,768, 6,288,220, 6,287,776, 6,297,006, and 6,291,193 which are hereby incorporated by reference in their entirety.

Detection Labels

To aid in detection and quantitation of nucleic acids amplified using the disclosed method, detection labels can be directly incorporated into amplified nucleic acids or can be coupled to detection molecules. As used herein, a detection label is any molecule that can be associated with amplified nucleic acid, directly or indirectly, and which results in a measurable, detectable signal, either directly or indirectly. Many such labels for incorporation into nucleic acids or coupling to nucleic acid probes are known to those of skill in the art. Examples of detection labels suitable for use in the disclosed method are radioactive isotopes, fluorescent molecules, phosphorescent molecules, enzymes, antibodies, and ligands.

Examples of suitable fluorescent labels include fluorescein isothiocyanate (FITC), 5,6-carboxymethyl fluorescein, Texas red, nitrobenz-2-oxa-1,3-diazol-4-yl (NBD), coumarin, dansyl chloride, rhodamine, amino-methyl coumarin (AMCA), Eosin, Erythrosin, BODIPY™, Cascade Blue™, Oregon Green™, pyrene, lissamine, xanthenes, acridines, oxazines, phycoerythrin, macrocyclic chelates of lanthanide ions such as quantum dye™, fluorescent energy transfer dyes, such as thiazole orange-ethidium heterodimer, and the cyanine dyes Cy3, Cy3.5, Cy5, Cy5.5 and Cy7. Examples of other specific fluorescent labels include 3-Hydroxypyrene 5,8,10-Tri Sulfonic acid, 5-Hydroxy Tryptamine (5-HT), Acid Fuchsin, Alizarin Complexon, Alizarin Red, Allophycocyanin, Aminocoumarin, Anthroyl Stearate, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, Blancophor FFG Solution, Blancophor SV, Bodipy F1, Brilliant Sulphoflavin FF, Calcien Blue, Calcium Green, Calcofluor RW Solution, Calcofluor White, Calcophor White ABT Solution, Calcophor White Standard Solution, Carbostyryl, Cascade Yellow, Catecholamine, Chinacrine, Coriphosphine O, Coumarin-Phalloidin, CY3.1 8, CY5.1 8, CY7, Dans (1-Dimethyl Amino Naphaline 5 Sulphonic Acid), Dansa (Diamino Naphtyl Sulphonic Acid), Dansyl NH—CH3, Diamino Phenyl Oxydiazole (DAO), Dimethylamino-5-Sulphonic acid, Dipyrrometheneboron Difluoride, Diphenyl Brilliant Flavine 7GFF, Dopamine, Erythrosin ITC, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow 5GF, Gloxalic Acid, Granular Blue, Haematoporphyrin, Indo-1, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nitrobenzoxadidole, Noradrenaline, Nuclear Fast Red, Nuclear Yellow, Nylosan Brilliant Flavin E8G, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Polyazaindacene Pontochrome Blue Black, Porphyrin, Primuline, Procion Yellow, Pyronine, Pyronine B, Pyrozal Brilliant Flavin 7GF, Quinacrine Mustard, Rhodamine 123, Rhodamine 5 GLD, Rhodamine 6G, Rhodamine B, Rhodarmine B 200, Rhodamine B Extra, Rhodamine BB, Rhodamine BG, Rhodamine WT, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulpho Rhodanine B Can C, Sulpho Rhodamine G Extra, Tetracycline, Thiazine Red R, Thioflavin S, Thioflavin TCN, Thioflavin 5, Thiolyte, Thiozol Orange, Tinopol CBS, True Blue, Ultralite, Uranine B, Uvitex SFC, Xylene Orange, and XRITC.

Preferred fluorescent labels are fluorescein (5-carboxyfluorescein-N-hydroxysuccinimide ester), rhodamine (5,6-tetramethyl rhodamine), and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. Other examples of fluorescein dyes include 6-carboxyfluorescein (6-FAM), 2′,4′,1,4,-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), and 2′-chloro-7′-phenyl-1,4-dichloro-6-carboxyfluorescein (VIC). Fluorescent labels can be obtained from a variety of commercial sources, including Amersham Pharmacia Biotech, Piscataway, N.J.; Molecular Probes, Eugene, Oreg.; and Research Organics, Cleveland, Ohio.

Additional labels of interest include those that provide for signal only when the probe with which they are associated is specifically bound to a target molecule, where such labels include: “molecular beacons” as described in Tyagi & Kramer, Nature Biotechnology (1996) 14:303 and EP 0 070 685 B1. Other labels of interest include those described in U.S. Pat. No. 5,563,037; WO 97/17471 and WO 97/17076.

Labeled nucleotides are a preferred form of detection label since they can be directly incorporated into the amplification products during synthesis. Examples of detection labels that can be incorporated into amplified nucleic acids include nucleotide analogs such as BrdUrd (5-bromodeoxyuridine, Hoy and Schimke, Mutation Research 290:217-230 (1993)), aminoallyldeoxyuridine (Henegariu et al., Nature Biotechnology 18:345-348 (2000)), 5-methylcytosine (Sano et al., Biochim. Biophys. Acta 951:157-165 (1988)), bromouridine (Wansick et al., J. Cell Biology 122:283-293 (1993)) and nucleotides modified with biotin (Langer et al., Proc. Natl. Acad. Sci. USA 78:6633 (1981)) or with suitable haptens such as digoxygenin (Kerkhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res., 22:3226-3232 (1994)). A preferred nucleotide analog detection label for DNA is BrdUrd (bromodeoxyuridine, BrdUrd, BrdU, BUdR, Sigma-Aldrich Co). Other preferred nucleotide analogs for incorporation of detection label into DNA are AA-dUTP (aminoallyl-deoxyuridine triphosphate, Sigma-Aldrich Co.), and 5-methyl-dCTP (Roche Molecular Biochemicals). A preferred nucleotide analog for incorporation of detection label into RNA is biotin-16-UTP (biotin-16-uridine-5′-triphosphate, Roche Molecular Biochemicals). Fluorescein, Cy3, and Cy5 can be linked to dUTP for direct labeling. Cy3.5 and Cy7 are available as avidin or anti-digoxygenin conjugates for secondary detection of biotin- or digoxygenin-labeled probes.

Detection labels that are incorporated into amplified nucleic acid, such as biotin, can be subsequently detected using sensitive methods well-known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4-methoxyspiro-[1,2,-dioxetane-3-2′-(5′-chloro)tricyclo[3.3.1.1.sup.3,7]decane]-4-yl)phenyl phosphate; Tropix, Inc.). Labels can also be enzymes, such as alkaline phosphatase, soybean peroxidase, horseradish peroxidase and polymerases, that can be detected, for example, with chemical signal amplification or by using a substrate to the enzyme which produces light (for example, a chemiluminescent 1,2-dioxetane substrate) or fluorescent signal.

Molecules that combine two or more of these detection labels are also considered detection labels. Any of the known detection labels can be used with probes, tags, and method to label and detect nucleic acid amplified using the disclosed method. Methods for detecting and measuring signals generated by detection labels are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary detection label coupled to the antibody. As used herein, detection molecules are molecules which interact with amplified nucleic acid and to which one or more detection labels are coupled.

Detection Probes

Detection probes are labeled oligonucleotides having sequence complementary to detection tags on amplified nucleic acids. The complementary portion of a detection probe can be any length that supports specific and stable hybridization between the detection probe and the detection tag. For this purpose, a length of about 10 to 35 nucleotides is preferred, with a complementary portion of a detection probe about 16 to 20 nucleotides long being most preferred. Detection probes can comprise any of the detection labels described above. Preferred labels are biotin and fluorescent molecules. A particularly preferred detection probe is a molecular beacon. Molecular beacons are detection probes labeled with fluorescent moieties where the fluorescent moieties fluoresce only when the detection probe is hybridized (Tyagi and Kramer, Nature Biotechnol. 14:303-309 (1995)). The use of such probes eliminates the need for removal of unhybridized probes prior to label detection because the unhybridized detection probes will not produce a signal. This is especially useful in multiplex assays.

Address Probes

An address probe is an oligonucleotide having a sequence complementary to address tags on primers. The complementary portion of an address probe can be any length that supports specific and stable hybridization between the address probe and the address tag. For this purpose, a length of about 10 to 35 nucleotides is preferred, with a complementary portion of an address probe about 12 to 18 nucleotides long being most preferred. An address probe can contain a single complementary portion or multiple complementary portions. Preferably, address probes are coupled, either directly or via a spacer molecule, to a solid-state support. Such a combination of address probe and solid-state support are a preferred form of solid-state detector.

Oligonucleotide Synthesis

Primers, detection probes, address probes, and any other oligonucleotides can be synthesized using established oligonucleotide synthesis methods. Methods to produce or synthesize oligonucleotides are well known in the art. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method. Solid phase chemical synthesis of DNA fragments is routinely performed using protected nucleoside cyanoethyl phosphoramidites (S. L. Beaucage et al. (1981) Tetrahedron Lett. 22:1859). In this approach, the 3′-hydroxyl group of an initial 5′-protected nucleoside is first covalently attached to the polymer support (R. C. Pless et al. (1975) Nucleic Acids Res. 2:773 (1975)). Synthesis of the oligonucleotide then proceeds by deprotection of the 5′-hydroxyl group of the attached nucleoside, followed by coupling of an incoming nucleoside-3′-phosphoramidite to the deprotected hydroxyl group (M. D. Matteucci et al. (1981) J. Am. Chem. Soc. 103:3185). The resulting phosphite triester is finally oxidized to a phosphorotriester to complete the internucleotide bond (R. L. Letsinger et al. (1976) J. Am. Chem. Soc. 9:3655). Alternatively, the synthesis of phosphorothioate linkages can be carried out by sulfurization of the phosphite triester. Several chemicals can be used to perform this reaction, among them 3H-1,2-benzodithiole-3-one, 1,1-dioxide (R. P. Iyer, W. Egan, J. B. Regan, and S. L. Beaucage, J. Am. Chem. Soc., 1990, 112, 1253-1254). The steps of deprotection, coupling and oxidation are repeated until an oligonucleotide of the desired length and sequence is obtained. Other methods exist to generate oligonucleotides such as the H-phosphonate method (Hall et al, (1957) J. Chem. Soc., 3291-3296) or the phosphotriester method as described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994). Other forms of oligonucleotide synthesis are described in U.S. Pat. Nos. 6,294,664 and 6,291,669.

The nucleotide sequence of an oligonucleotide is generally determined by the sequential order in which subunits of subunit blocks are added to the oligonucleotide chain during synthesis. Each round of addition can involve a different, specific nucleotide precursor or a mixture of one or more different nucleotide precursors. In general, degenerate or random positions in an oligonucleotide can be produced by using a mixture of nucleotide precursors representing the range of nucleotides that can be present at that position. Thus, precursors for A and T can be included in the reaction for a particular position in an oligonucleotide if that position is to be degenerate for A and T. Precursors for all four nucleotides can be included for a fully degenerate or random position. Completely random oligonucleotides an be made by including all four nucleotide precursors in every round of synthesis. Degenerate oligonucleotides can also be made having different proportions of different nucleotides. Such oligonucleotides can be made, for example, by using different nucleotide precursors, in the desired proportions, in the reaction.

Many of the oligonucleotides described herein are designed to be complementary to certain portions of other oligonucleotides or nucleic acids such that stable hybrids can be formed between them. The stability of these hybrids can be calculated using known methods such as those described in Lesnick and Freier, Biochemistry 34:10807-10815 (1995), McGraw et al., Biotechniques 8:674-678 (1990), and Rychlik et al., Nucleic Acids Res. 18:6409-6412 (1990).

Polymerases

In a preferred embodiment, Φ29 DNA polymerase is used in the nucleic acid amplification in yeast as described in the Examples which follow. In addition to Φ29 DNA polymerase, other DNA polymerases may be used in methods of the invention. Any DNA polymerase that performs at least one functional activity of Φ29. DNA polymerase may be used. For example, polymerases having high fidelity and processivity. Examples of suitable DNA polymerases include those of phages Φ15, PZA, PZE, BS32, B103, Nf and M2 (see Meijer et al., Microbiology and Molecular Biology Reviews, 65:261-287, 2001), Bst large fragment DNA polymerase (Exo(−) Bst; Aliotta et al., Genet. Anal. (Netherlands) 12:185-195 (1996)) and exo(−)Bca DNA polymerase (Walker and Linn, Clinical Chemistry 42:1604-1608 (1996)). Other useful polymerases include phage M2 DNA polymerase (Matsumoto et al., Gene 84:247 (1989)), phage ΦPRD1 DNA polymerase (Jung et al., Proc. Natl. Acad. Sci. USA 84:8287 (1987)), exo(−)VENT™ DNA polymerase (Kong et al., J. Biol. Chem. 268:1965-1975 (1993)), Klenow fragment of DNA polymerase I (Jacobsen et al., Eur. J. Biochem. 45:623-627 (1974)), T5 DNA polymerase (Chatterjee et al., Gene 97:13-19 (1991)), Sequenase (U.S. Biochemicals), PRD1 DNA polymerase (Zhu and Ito, Biochim. Biophys. Acta. 1219:267-276 (1994)), and T4 DNA polymerase holoenzyme (Kaboord and Benkovic, Curr. Biol. 5:149-157 (1995)). However, Φ29 DNA polymerase is most preferred.

Amplification of Genes for Diagnostic and Therapeutic Purposes

In accordance with the invention nucleic acid molecules derived from cells that have been infected with an infectious organism or from a patient suffering from or susceptible to a disease are amplified by the disclosed method. Infectious disease almost invariably results in the acquisition of foreign nucleic acids, which could be targeted using this technology. Specific targets could be viral, e.g. HIV (virus or provirus) or bacterial, e.g. multi-drug resistant bacteria e.g. TB, fungal or protoazoan.

Preferably, the primers identified by any one of SEQ ID NO's 1-8 or homologous variants thereof, will hybridize (bind) to a target sequence, particularly a target oligonucleotide of a cancer cell or an infectious agent such as a viral, bacterial, fungal or protozoan agent including those agents and sequences disclosed herein, under stringency conditions as may be assessed in vitro. Such conditions are disclosed and defined below.

The invention may be used to amplify protein coding genes as well as non-protein coding genes. Examples of non-protein coding genes include genes that encode ribosomal RNAs, transfer RNAs, small nuclear RNAs, small cytoplasmic RNAs, telomerase RNA, RNA molecules involved in DNA replication, chromosomal rearrangement and the like.

In another preferred embodiment, abnormal or cancer cells are targeted by the primers for amplification. For example, many malignancies are associated with the presence of foreign DNA, e.g. Bcr-Ab1, Bcl-2, HPV, and these provide unique molecular targets to permit selective malignant cell targeting. The approach can be used to target single base substitutions (e.g. K-ras, p53) or methylation changes. However, proliferation of cancer cells may also be caused by previously unexpressed genes. These gene sequences can be targeted. In other instances, transposons can be the cause of such deregulation and transposon sequences can be targeted, e.g. Tn5.

In another preferred embodiment, the genes from cancer cells can be amplified to for use in screening for candidate therapeutic agents. Genes which are over expressed in a cancer cell can be identified as compared to normal cells. For example, Expressed Sequenced Tags (ESTs), can be used to identify nucleic acid molecules which are over expressed in a cancer cell [expressed sequence tag (EST) sequencing (Celis, et al., FEBS Lett., 2000, 480, 2-16; Larsson, et al., J. Biotechnol., 2000, 80, 143-57)]. ESTs from a variety of databases can be identified. For example, preferred databases include, for example, Online Mendelian Inheritance in Man (OMIM), the Cancer Genome Anatomy Project (CGAP), GenBank, EMBL, PIR, SWISS-PROT, and the like. OMIM, which is a database of genetic mutations associated with disease, was developed, in part, for the National Center for Biotechnology Information (NCBI). OMIM can be accessed through the world wide web of the Internet, at, for example, ncbi.nlm.nih.gov/Omim/. CGAP, which is an interdisciplinary program to establish the information and technological tools required to decipher the molecular anatomy of a cancer cell. CGAP can be accessed through the world wide web of the Internet, at, for example, ncbi.nlm.nih.gov/ncicgap/. Some of these databases may contain complete or partial nucleotide sequences. In addition, alternative transcript forms can also be selected from private genetic databases. Alternatively, nucleic acid molecules can be selected from available publications or can be determined especially for use in connection with the present invention.

Alternative transcript forms can be generated from individual ESTs which are within each of the databases by computer software which generates contiguous sequences. In another embodiment of the present invention, the nucleotide sequence of the target nucleic acid molecule is determined by amplifying a plurality of overlapping ESTs. The EST database (dbEST), which is known and available to those skilled in the art, comprises approximately one million different human mRNA sequences comprising from about 500 to 1000 nucleotides, and various numbers of ESTs from a number of different organisms. dbEST can be accessed through the world wide web of the Internet, at, for example, ncbi.nlm.nih.gov/dbEST/index.html. ESTs have applications in the discovery of new genes, mapping of genomes, and identification of coding regions in genomic sequences. Another important feature of EST sequence information that is becoming rapidly available is tissue-specific gene expression data. This can be extremely useful in targeting selective gene(s) for therapeutic intervention. Since EST sequences are relatively short, they must be assembled in order to provide a complete sequence. Because every available clone is sequenced, it results in a number of overlapping regions being reported in the database. The end result is the elicitation of alternative transcript forms from, for example, normal cells and cancer cells.

Assembly of overlapping ESTs extended along both the 5′ and 3′ directions results in a full-length “virtual transcript.” The resultant virtual transcript may represent an already characterized nucleic acid or may be a novel nucleic acid with no known biological function. The Institute for Genomic Research (TIGR) Human Genome Index (HGI) database, which is known and available to those skilled in the art, contains a list of human transcripts.

The present invention provides a method of amplifying large numbers and sizes of genes for diagnosis, identification of disease causing genes, and for identifying compounds with therapeutic potential. For example, genes which are overexpressed by cancer cells as compared to normal cells, for example, genes expressed at least 5 fold greater in pancreatic cancers compared to normal tissues can be identified and amplified using the disclosed method. Amplified genes can be analyzed by Serial Analysis of Gene Expression (SAGE), which is based on the identification of and characterization of partial, defined sequences of transcripts corresponding to gene segments [SAGE (serial analysis of gene expression) (Madden, et al., Drug Discov. Today, 2000, 5, 415-425)]. These defined transcript sequence “tags” are markers for genes which are expressed in a cell, a tissue, or an extract, for example. For example, a tag as short as 6-7 bp may be sufficient for distinguishing transcripts in yeast. The full length genes can be identified by matching the tag to a gene data base member, or by using the tag sequences as probes to physically isolate previously unidentified genes from cDNA libraries. The methods by which genes are isolated from libraries using DNA probes are well known in the art. See, for example, Veculescu et al., Science 270: 484 (1995), and Sambrook et al. (1989), MOLECULAR CLONING: A LABORATORY MANUAL, 2nd ed. (Cold Spring Harbor Press, Cold Spring Harbor, N.Y.). Once a gene or transcript has been identified, either by matching to a data base entry, or by physically hybridizing to a cDNA molecule, the position of the hybridizing or matching region in the transcript can be determined. If the tag sequence is not in the 3′ end, immediately adjacent to the restriction enzyme used to generate the SAGE tags, then a spurious match may have been made. Confirmation of the identity of a SAGE tag can be made by comparing transcription levels of the tag to that of the identified gene in certain cell types.

Analysis of gene expression is not limited to the above methods but can include any method known in the art. All of these principles may be applied independently, in combination, or in combination with other known methods of sequence identification.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U.S.A., 2000, 97, 1976-81), protein arrays and proteomics (Celis, et al., FEBS Lett., 2000, 480, 2-16; Jungblut, et al., Electrophoresis, 1999, 20, 2100-10), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), FISH (fluorescent in situ hybridization) techniques (Going and Gusterson, Eur. J. Cancer, 1999, 35, 1895-904) and mass spectrometry methods (reviewed in (Comb. Chem. High Throughput Screen, 2000, 3, 235-41)).

In yet another aspect, the method provides for the amplification of target genes that are variants and are useful for diagnosing and identifying agents for treatment of cancer. For example, p53 mutants are well known in a variety of tumors. A “variant” is an alternative form of a gene. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

Sequence similarity searches can be performed manually or by using several available computer programs known to those skilled in the art. Preferably, Blast and Smith-Waterman algorithms, which are available and known to those skilled in the art, and the like can be used. Blast is NCBI's sequence similarity search tool designed to support analysis of nucleotide and protein sequence databases. Blast can be accessed through the world wide web of the Internet, at, for example, ncbi.nlm.nih.gov/BLAST/. The GCG Package provides a local version of Blast that can be used either with public domain databases or with any locally available searchable database. GCG Package v9.0 is a commercially available software package that contains over 100 interrelated software programs that enables analysis of sequences by editing, mapping, comparing and aligning them. Other programs included in the GCG Package include, for example, programs which facilitate RNA secondary structure predictions, nucleic acid fragment assembly, and evolutionary analysis. In addition, the most prominent genetic databases (GenBank, EMBL, PIR, and SWISS-PROT) are distributed along with the GCG Package and are fully accessible with the database searching and manipulation programs. GCG can be accessed through the Internet at, for example, http://www.gcg.com/. Fetch is a tool available in GCG that can get annotated GenBank records based on accession numbers and is similar to Entrez. Another sequence similarity search can be performed with GeneWorld and GeneThesaurus from Pangea. GeneWorld 2.5 is an automated, flexible, high-throughput application for analysis of polynucleotide and protein sequences. GeneWorld allows for automatic analysis and annotations of sequences. Like GCG, GeneWorld incorporates several tools for homology searching, gene finding, multiple sequence alignment, secondary structure prediction, and motif identification. GeneThesaurus 1.0™ is a sequence and annotation data subscription service providing information from multiple sources, providing a relational data model for public and local data.

Another alternative sequence similarity search can be performed, for example, by BlastParse. BlastParse is a PERL script running on a UNIX platform that automates the strategy described above. BlastParse takes a list of target accession numbers of interest and parses all the GenBank fields into “tab-delimited” text that can then be saved in a “relational database” format for easier search and analysis, which provides flexibility. The end result is a series of completely parsed GenBank records that can be easily sorted, filtered, and queried against, as well as an annotations-relational database.

In another preferred embodiment, the method disclosed herein can be used to amplify large numbers of genes for screening in libraries. Amplified genes can be identified from individuals suffering form or susceptible to such as autoimmune disease; hypersensitivity to allergans; organ rejection; inflammation; and the like. Examples of inflammation associated with conditions such as: adult respiratory distress syndrome (ARDS) or multiple organ injury syndromes secondary to septicemia or trauma; reperfusion injury of myocardial or other tissues; acute glomerulonephritis; reactive arthritis; dermatoses with acute inflammatory components; acute purulent meningitis or other central nervous system inflammatory disorders; thermal injury; hemodialysis; leukapheresis; ulcerative colitis; Crohn's disease; necrotizing enterocolitis; granulocyte transfusion associated syndromes; and cytokine-induced toxicity. Examples of autoimmune diseases include, but are not limited to psoriasis, Type I diabetes, Reynaud's syndrome, autoimmune thyroiditis, EAE, multiple sclerosis, rheumatoid arthritis and lupus erythematosus

Kits

In yet another aspect, the invention provides kits for amplifying gene sequences of infectious disease organism, cancer, autoimmune diseases and the like. For example, the kits can be used to target any desired nucleic sequence, such as an HPV sequence. The kits of the invention have many applications. For example, the kits can be used to idebtify cells infected with a virus, or if the cells are at different stages of a tumor.

In one embodiment, a kit comprises: an isolated yeast cell; zymolase; a vector; a reaction mixture comprising a DNA polymerase; primers identified by any one of SEQ ID NO's 1-8.

Optionally, the kit can further comprise instructions for suitable operational parameters in the form of a label or a separate insert. For example, the kit may have standard instructions informing a physician or laboratory technician to prepare the yeast cells In another example, the kit may have instructions for optimizing the amplification of a particular gene from a cell infected with virus, fungus and the like.

Biological Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22:1859-1862, 1981, and Matteucci et al., J. Am. Chem. Soc., 103:3185, 1981. Chemical synthesis of nucleic acids can be performed, for example, on commercial automated oligonucleotide synthesizers.

The invention has been described in detail with reference to preferred embodiments thereof. However, it will be appreciated that those skilled in the art, upon consideration of this disclosure, may make modifications and improvements within the spirit and scope of the invention. The following non-limiting examples are illustrative of the invention.

### EXAMPLES

Materials and Methods

Plasmid construction: pBOK is derived from the yeast two-hybrid vector pPC97 (Chevray and Nathans, 1992; Chem et al., 2001) carrying the GAL4 DNA binding domain by swapping the Kpn I fragment with the corresponding region of the pDBLeu vector that contains the kanamycin resistance gene (Invitrogen, Carlsbad Calif.). XA21K was amplified using PCR, verified by sequencing, and cloned into pBOK to create pBOK-XA21K. Primers used to amplify XA21K include: primer 822-29, 5′-AGGTCGACCCCGGGAATGAAAGGCCACCCATT-3′; primer 822-20, 5′-GGGGTACCGGATCCTCAAAATTCAAGGCTCCCACCTTCAA-3′.

pPC86-UK was identified from two-hybrid screening of a rice cDNA library (Yin et al., 1997) using the kinase domain of the rice resistance protein XA21 (XA21K) (Song et al., 1995) as bait.

RCA reaction and restriction digestion of the RCA-amplified DNA: The RCA reactions were performed using the TempliPhi 100 Amplification kit (Amersham Biosciences, Piscataway, N.J.). Briefly, ⅓-½ of each single yeast colony was picked using toothpicks and suspended into 5 μL of sample buffer (Amersham Biosciences, Piscataway, N.J.). For enzymatic treatment, 2 units of zymolase (Zymo Research, Orange, Calif.) were added and the reaction mixtures were incubated at 37° C. for 15 min. For freeze/thaw treatment, the resuspended cells were frozen at −80° C. for 5 min and then thawed at room temperature for 5 min. For amplification from glycerol stocks, 1 μL of yeast stocks was mixed with 5 μL of sample buffer.

The resuspended cells were incubated at 95° C. for 3 min and then cooled down to 4° C. 5 μL of reaction buffer and 0.2 μL of the Φ29 DNA polymerase (Amersham Biosciences, Piscataway, N.J.) were added to the denatured cell suspensions. The mixtures were incubated at 30° C. for 6-18 hours for amplification and then heated at 65° C. to stop the reactions.

Restriction digestion of the RCA products was performed in 15 μL volumes. 3 μL of amplified DNA were transferred to a new test tube and incubated with restriction enzymes in the appropriate buffer at 37° C. for 4 hours. The reactions were stopped by heating the samples to 65° C. for 20 min.

Bacterial and yeast transformation: Transformation of E. coli XL1-blue cells (Stratagene, La Jolla, Calif.) was carried out by electroporation using a Cell-Portator (Invitrogen, Carlsbad, Calif.) according to manufacturer's instructions.

The strain CG-1945 was used as the yeast host in this study. Competent cells were prepared using the Yeast Competent Cell Preparation kit (Zymo Research, Orange, Calif.). Transformation of the competent cells was carried out according to manufacturer's instructions (Zymo Research, Orange, Calif.).

DNA sequencing of RCA products: Sequencing templates were prepared directly from yeast and bacterial cultures using TempliPhi™ DNA Sequencing Template Amplification method as specified by the manufacturer (Amersham Biosciences, Piscataway, N.J.). Dye terminator DNA sequencing reactions utilized ET-Terminators from Amersham Biosciences. Sequence reactions were desalted by ethanol precipitation, dried, and resuspended in 10 μL of 0.06% aqueous solution of Seakem Gold agarose (Cambrex, Rockland, Me.) prior to electrokinetic injection. All DNA sequencing was performed on capillary array DNA sequencing units (MegaBACE 1000, Amersham Biosciences, Piscataway, N.J.) and base-called using PHRAP (Ewing et al., 1998; Ewing and Green, 1998).

The primers GAL4-DB (5′-TCATCGGAAGAGAGTAG-3′) and SSO₂₀ (5′-AGGGATGTTTAATACCACTAC-3′) were used to sequence the RCA-amplified pBOK-XA21K and pPC86-UK from yeast cells, respectively. The primers used to sequence the insert of pBOK-XA21K and pPC86-UK include:

| | |
|---|---|
| GAL4-DB, | |
| TADC1 | (5′-TTGATTGGAGACTTGACC-3′), |
| SSO20, | |
| UK-Seq-1 | (5′-CCCAACCAAGAAGTATGCTGA-3′), |
| UK-Seq-2 | (5′-TCACAGGGAAGGAGACAATGGCA-3′) |
| and | |
| UK-Seq-3 | (5′-ATGAAATCATGTGTTGTTGTGGCA-3′). |

Yeast two-hybrid assays for protein-protein interactions: Yeast cells (CG-1945) transformed with RCA-amplified pBOK-XA21K/pPC86-UK or pBOK-XA21K-K736E/pPC86-UK were plated on SD minimal medium (MATCHMAKER GAL4 Two-Hybrid User Manual, Clontech, Palo Alto, Calif.) lacking leucine and tryptophan to select for the presence of these two plasmids in the yeast cells. As a control, the wild-type pBOK-XA21K/pPC86-UK and pBOK-XA21K-K736E/pPC86-UK plasmids were employed to carry out similar transformation. After incubation at 30° C. for 2-3 days, the colonies were streaked onto SD minimal medium containing 25 mM 3-Amino-1,2,4-triazole, but lacking leucine, tryptophan and histidine to select for the cells containing interacting proteins. After 3-5 days of incubation at 30° C., the colonies were photographed.

### Example 1

### Amplification of Plasmid DNA from Yeast

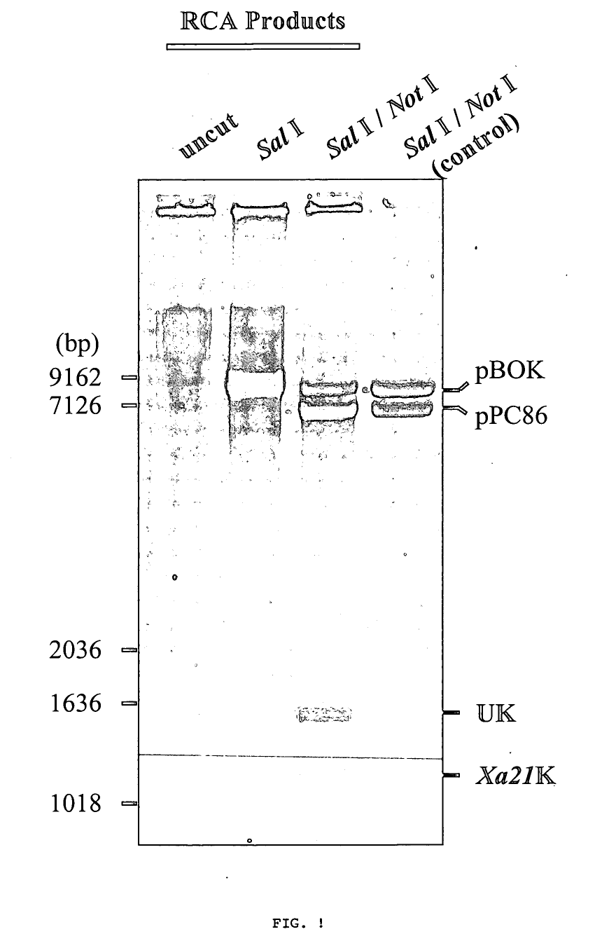

Yeast stain CG-1945 carrying both pBOK-XA21K and pPC86-UK constructs was chosen to perform RCA. These two shuttle plasmids can be distinguished by antibiotic selection in E. coli (pBOK-XA21K: kanamycin^{R}, pPC86-UK: ampicillin^{R}), by complementation in yeast (pBOK-XA21K: LEU2, pPC86-UK: TRP1), and by their restriction patterns after digestion with the enzymes SalI and NotI (pBOK-XA21K: 8.5 kb+1.2 kb, pPC86-UK: 7.0 kb+1.6 kb). To carry out amplification, 3 day-old single colonies were picked directly from plates. After a rapid DNA isolation procedure (described below) and RCA amplification for 12 hours at 30° C., 1-3 μg of DNA in a broad size range was amplified (FIG. 1). Single yeast colonies were picked and amplified in 10 μL volumes as described in the Materials and Methods section above. One μl of the amplified DNA was digested with indicated enzymes. Restriction digestion of the pBOK-XA21K and pPC86-UK plasmids prepared by conventional methods was performed as a control. The samples were resolved by agarose gel electrophoresis.

To confirm that the amplified products contained pBOK-XA21K and pPC86-UK, restriction digestion with the single-cut enzyme SalI was performed. A major band of 9 kb was obtained (FIG. 1). Double-digestion with SalI and NotI created four bands that were identical in size to the products of pBOK-XA21K and pPC86-UK cleaved by the same enzymes. These results indicated that the RCA products contained pBOK-XA21K and pPC86-UK, and that the amplified DNA was concatemeric. In addition to pBOK-XA21K and pPC86-UK that comprised the majority of the amplified product, some background, presumably amplified from yeast chromosomal DNA, was also observed.

### Example 2

### Optimization of Template Preparation and Plasmid RCA Conditions

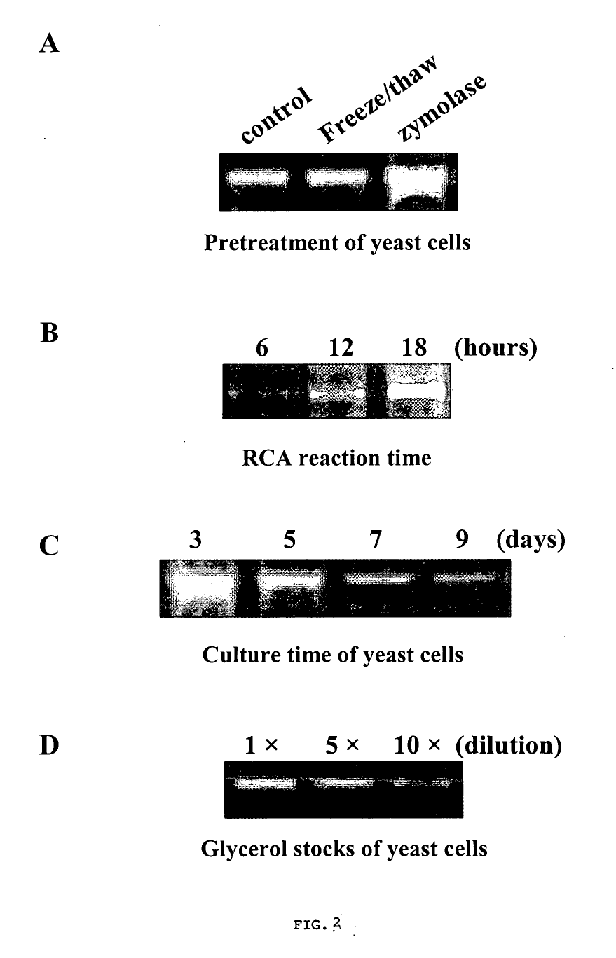

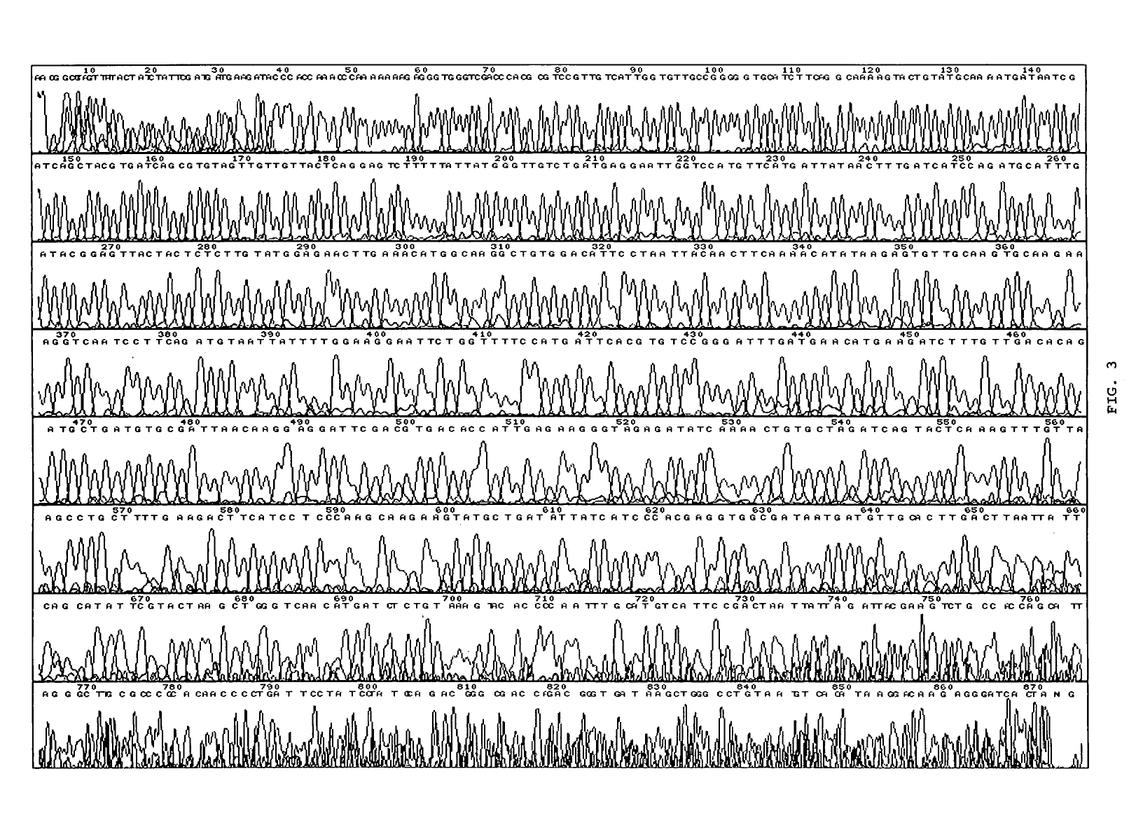

The template preparation and RCA reactions were optimized using yeast cells containing a single plasmid (pBOK-XA21K). The amplified products were resolved by agarose gel electrophoresis (FIG. 2). All the reactions were incubated at 30° C. for 12 hours except for panel (B). Freeze/thaw treatments have been used to lyse yeast cells in β-galactosidase filter assays (MATCHMAKER GAL4 Two-Hybrid User Manual, Clontech, Palo Alto, Calif.). However, these treatments did not result in an increase in the amounts of amplified products (FIG. 2A). In contrast, incubation of cells with zymolase (Zymo Research, Orange, Calif.), an enzyme that hydrolyzes glucose polymers of yeast cell walls at β-1,3-glucan linkages, significantly increased the yields of RCA products. Following zymolase treatment, amounts of amplified DNA increased with RCA reaction time (FIG. 2B). Six hours after RCA reaction, the amplified products can be detected by gel electrophoresis.

Amplification efficiency using yeast cells cultured for different time periods and cells from glycerol stocks was tested. Younger cells yielded better templates for RCA. A linear decrease in the amounts of amplified products was observed with an increase in culture time during the 9 day period examined (FIG. 2C). For example, the yield from 3 day-old yeast cells is 10-fold higher than that amplified from 9 day-old cells. In addition, the pBOK-XA21K DNA was also amplified from yeast glycerol stocks (FIG. 2D). Three days of growth were optimum.

### Example 3

### DNA Sequencing of RCA Products

RCA-amplified pBOK-XA21K and pPC86-UK were directly subjected to DNA sequence using Dye terminator DNA sequencing reactions (Amersham Biosciences, Piscataway, N.J.) with primers SSO20 (that specifically recognizes pPC86-UK) and GAL4-DB (that specifically recognizes pBOK-XA21K). After the reactions, the samples were desalted by ethanol precipitation and then electrokinetically injected in the capillary DNA sequencer. Up to 600 bases of high-quality sequence was typically obtained in a single run (FIG. 3). Thus, RCA-amplified plasmids were excellent templates for DNA sequencing.

### Example 4

### Retransformation of the RCA Amplified DNA into Yeast for Protein-Protein Interaction Assays

pBOK-XA21K encodes a fusion protein of the yeast GAL4 DNA binding domain and XA21K, whereas pPC86-UK encodes the GAL4 activation domain and the rice uridine kinase (UK) fusion. It has been shown previously that XA21K interacts with UK in the yeast two-hybrid system, but not with a XA21K mutant, XA21-K736E. To determine whether the RCA products function as their templates after amplification and transformation, two pairs of plasmids (pBOK-XA21K/pPC86-UK and pBOK-XA21K-K736E/pPC86-UK) prepared by RCA and by the conventional rescue methods respectively, were transformed into yeast cells.

Yeast cells transformed with the indicated constructs were grown on selective medium. Colonies capable of growing on the SD medium without leucine and tryptophan showed the presence of the transformed plasmids, whereas colonies growing on the SD medium lacking leucine, tryptophan and histidine indicated activation of the reporter gene His3 resulting from the interactions of the XA21K and UK fusion proteins in the yeast cells. The plasmid DNA used for transformation was isolated from E. coli using conventional methods.

By using standard procedures, an efficiency of 4.6×10³ transformants/μg DNA was achieved for the RCA-amplified pPC86-UK (Table 1). To identify cells with the interactions between the XA21K and UK proteins, the transformants were selected on SD minimal medium without leucine, tryptophan and histidine. It was found that the RCA-amplified DNA functioned similar to its template in all aspects tested, including LEU2, TRP1, HIS3 selections and the XA21K-UK specific interactions.

TABLE 1
Transformation^{a} efficiency of yeast with
RCA DNA (pBOK-XA21K + pPC86-UK)

| Strain | CG1945 | CG1945 (pBOK-XA21K |
|---|---|---|
| Efficiency | ^{b}2 | ^{b}4.6 × 10³ |
| (transformants/μg DNA) | | |

^{a}Yeast cells containing the plasmids pBOK-XA21K + pPC86-UK were used as template for RCA. The amplified DNA was transformed into the indicated yeast cells using the method described in Example 1.
^{b}Transformants were selected on SD minimal medium lacking leucine and tryptophan.

Accuracy of RCA products: To estimate the error rate of RCA, plasmid DNA was isolated from 20 randomly picked bacterial clones transformed with the RCA-amplified pBOK-XA21K and pPC86-UK. The coding region of Xa21K (1042 bp) and UK (1486 bp) was completely sequenced. Only one mutation (T to C) was found in the coding region of the UK gene. The mutation, however, causes no amino acid substitutions. These observations were consistent with the results from the functional assays described above, indicating that the RCA-based plasmid rescue from yeast is functionally reliable.

In another example of an RCA-based technique for amplifying DNA plasmid DNA from yeast cells, the amplification products can be restriction digested with a single-cut restriction enzyme and self-ligated before transforming E. coli and yeast cells.

### Example 4

### High-Throughput System Using RCA

Among the methods developed to detect protein-protein interactions, yeast two-hybrid screening is simple, affordable, and sensitive. To reduce false positives, it is important to show that the identified interactions are reproducible by retransforming yeast with the constructs encoding the DNA binding (BD)1 and activation fusions (also known as bait and prey, respectively). As a negative control, the prey is often tested for no interactions with unrelated baits, including the vector encoding empty BD. It is difficult, however, to include these steps in a large-scale analysis, mainly because the plasmid DNA isolated from yeast requires propagation in Escherichia coli to produce a sufficient amount of DNA for the subsequent experiments.

We have recently co-amplified bait and prey plasmids from yeast cells using rolling circle amplification (RCA). Here, it is shown that the bait construct can be eliminated from the yeast transformants by using cycloheximide counterselection. Moreover, an RCA-based high-throughput system for characterization and verification of candidate interactors identified from initial yeast two-hybrid screening, is we demonstrated.

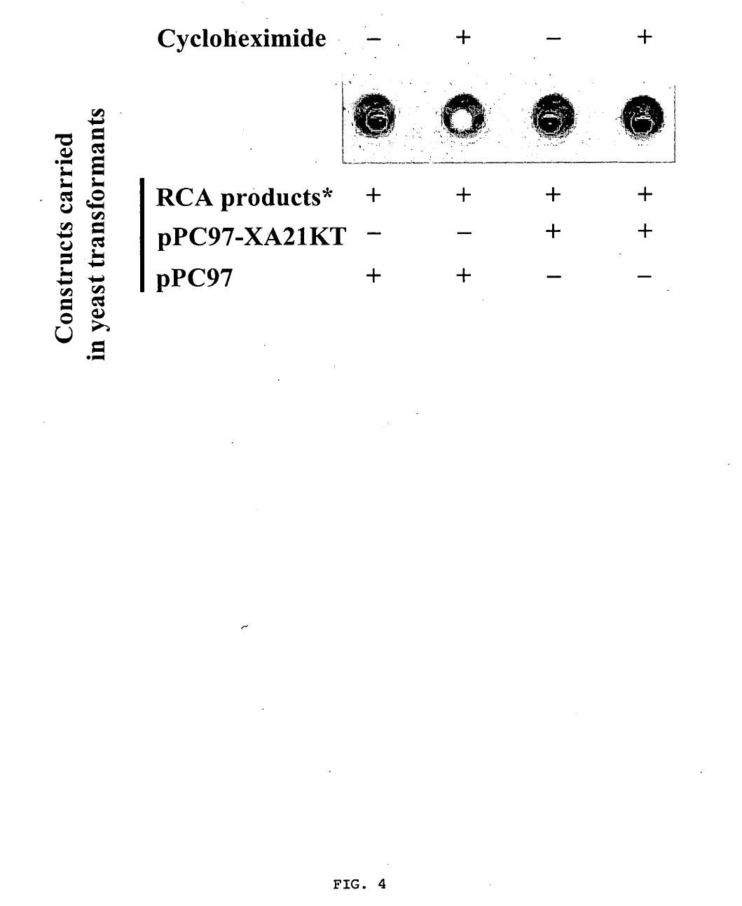

To test for lack of interactions of the prey with unrelated baits, cycloheximide counterselection was adopted to exclude the original bait in the RCA products. The bait vector pDBLeu carries the yeast gene CYH2 that makes the yeast strain MaV203 (MATα, leu2-3, 112 trp1-901, his3Δ200, ade2-101, gal4Δ, gal80Δ, SPAL10::URA3, GAL1::lacZ HIS3_{UAS GAL1}::HIS3@LYS2, can1^{R}, cyh2^{R}) susceptible to the inhibitor. The RCA-amplified bait pDBLeu-XA21KT and prey pPC86-UK were retransformed into the MaV203 cells containing either empty pPC97 vector or pPC97—XA21KT. The two-hybrid vector pPC97 is similar to pDBLeu except for the lack of 45 the CYH2 gene. Yeast transformants were plated on selective medium (SD/-Leu-Trp) with or without cycloheximide. Since XA21KT interacts with UK, the pPC97—XA21KT-containing yeast cells, transformed with the RCA-amplified pDBLeu-XA21KT and pPC86— UK, turned blue in the β-galactosidase assays regardless of the cycloheximide counter selection (FIG. 4). When transformed with the RCA products, the cells carrying the pPC97 empty vector showed no detectable β-galactosidase activity in the presence of cycloheximide but a strong P-galactosidase activity in the absence of the inhibitor. These results indicate that the cycloheximide counter selection can efficiently select against the presence of pDBLeu-XA21KT in the RCA products.

RCA and cycloheximide counterselection was integrated into yeast two-hybrid analysis. A rice cDNA library, constructed using the pPC86 vector, was screened using pDBLeu-XA21KT. Ninety-six candidate colonies were randomly picked from the initial screening and grown in a 96-well plate containing liquid selective medium (SD/-Trp-Leu-His+10 mM 3-AT). Cell lysates were tested for use as RCA templates. Two hundred microliters of yeast cells from the master plate were harvested, resuspended in 100 μL of TE supplemented with 1.5 μL of zymolase (Zymo Research, CA, USA), and incubated at 37° C. for 1 h. In addition, plasmid DNA, isolated from the same volume of the above cells using the Yeast Plasmid Miniprep kit (Zymo Research, CA, USA), was used as template for RCA. The results obtained show that both bait and prey can be directly amplified from cell lysates in a 96-well format using a similar procedure.

To obtain a better yield, a prolonged incubation time (30 h) was used. When using isolated plasmid DNA as templates, more than fourfold higher yields were obtained with only 18-h reactions. The amplification of 96 samples with a 100% success rate was achieved. The restriction pattern of RCA products amplified from both cell lysates and isolated plasmid DNA is identical, suggesting that the same plasmid DNA was amplified.

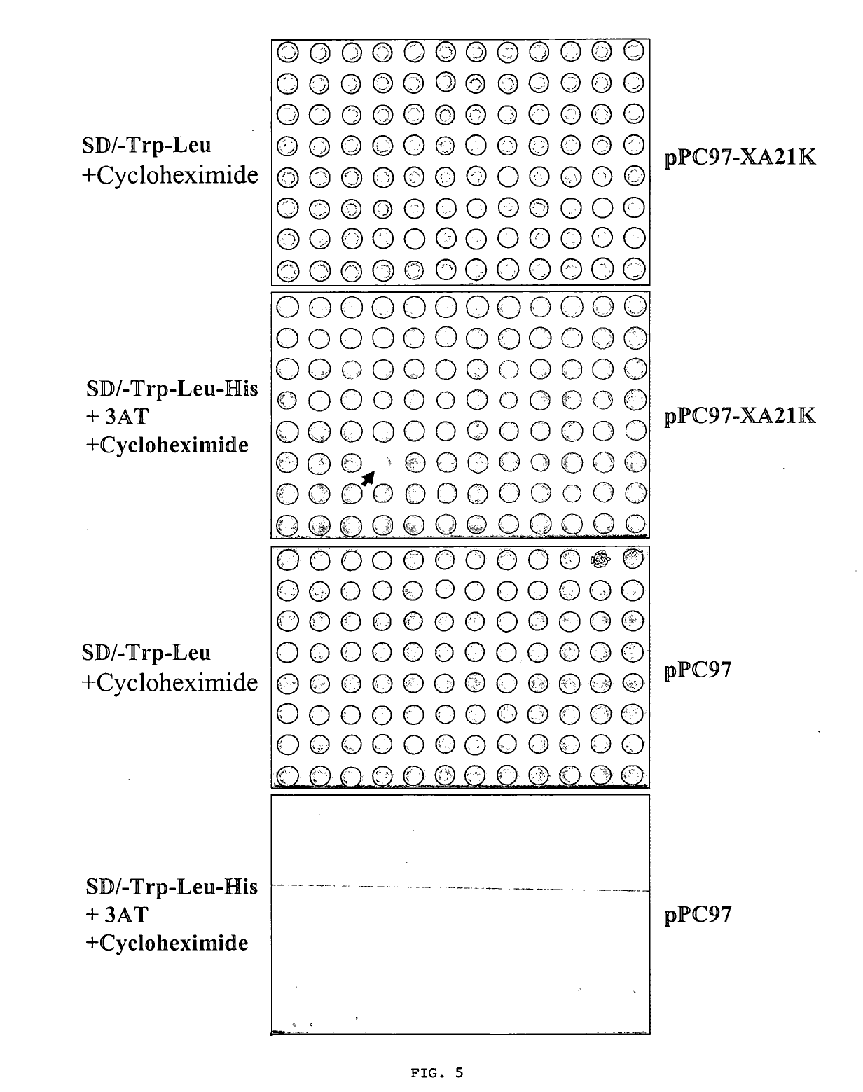

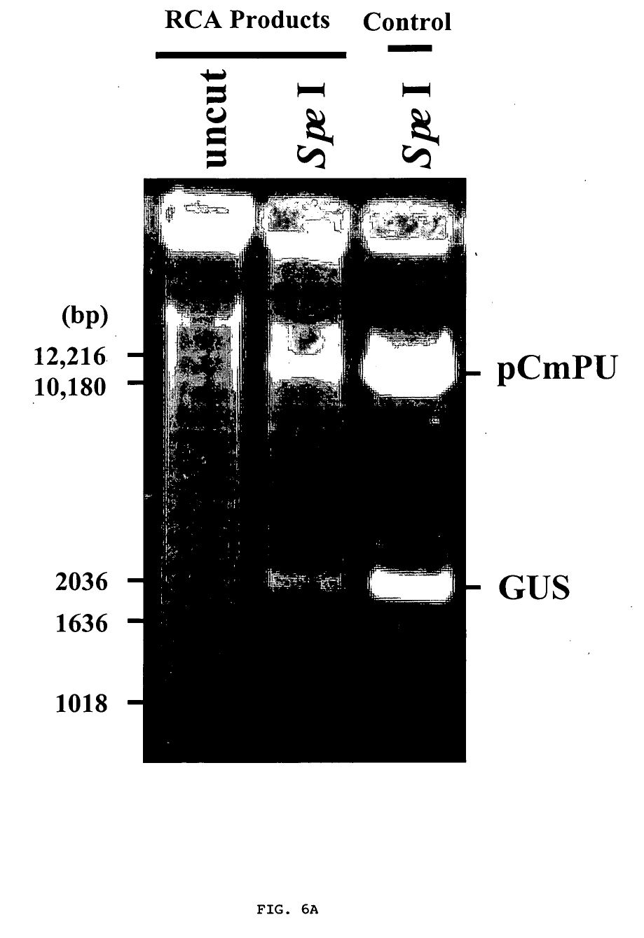

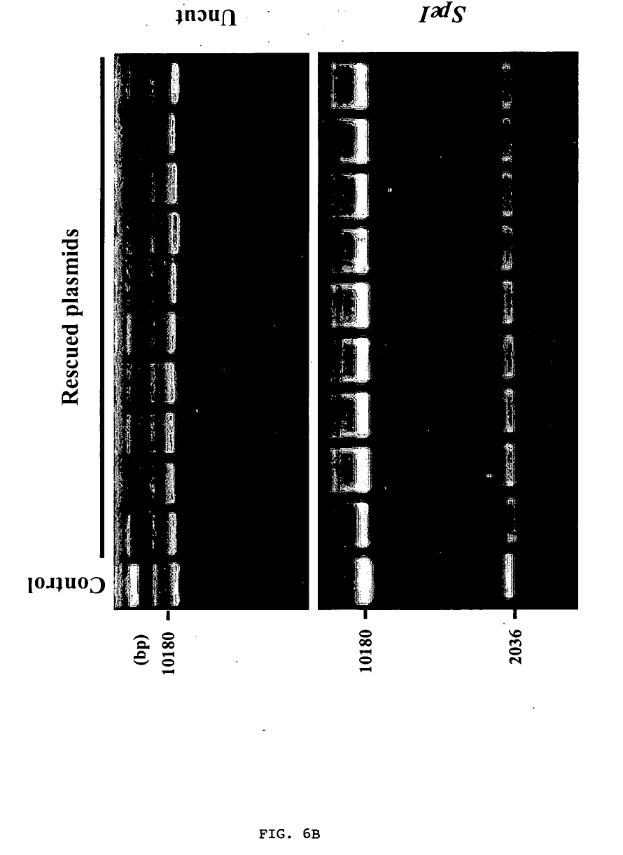

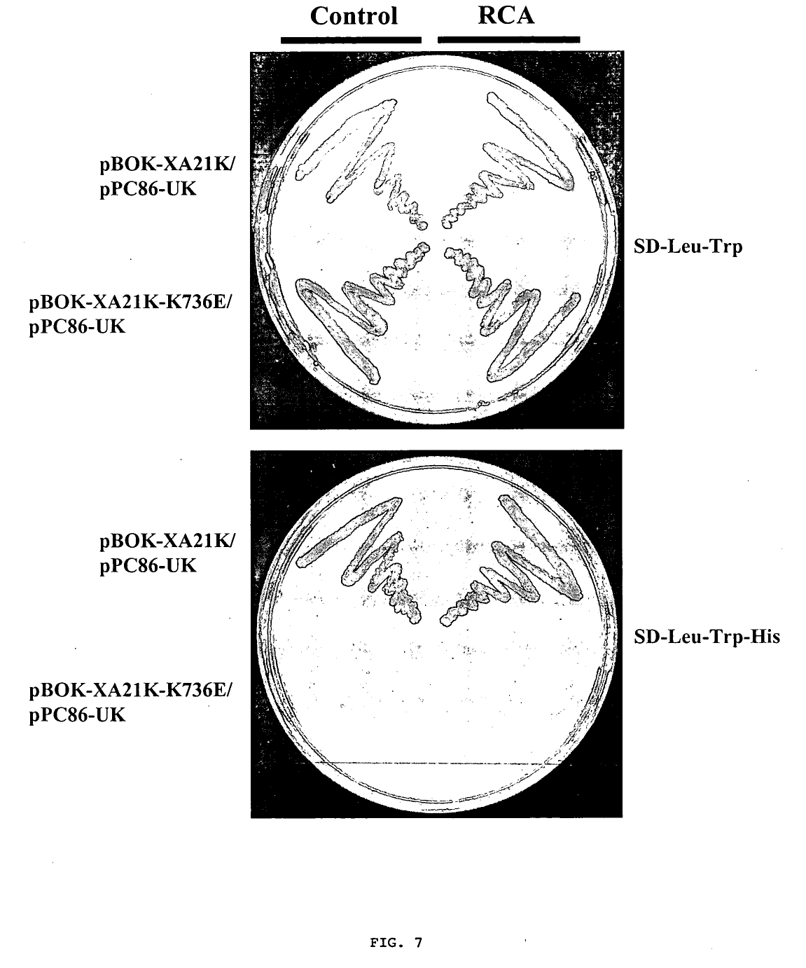

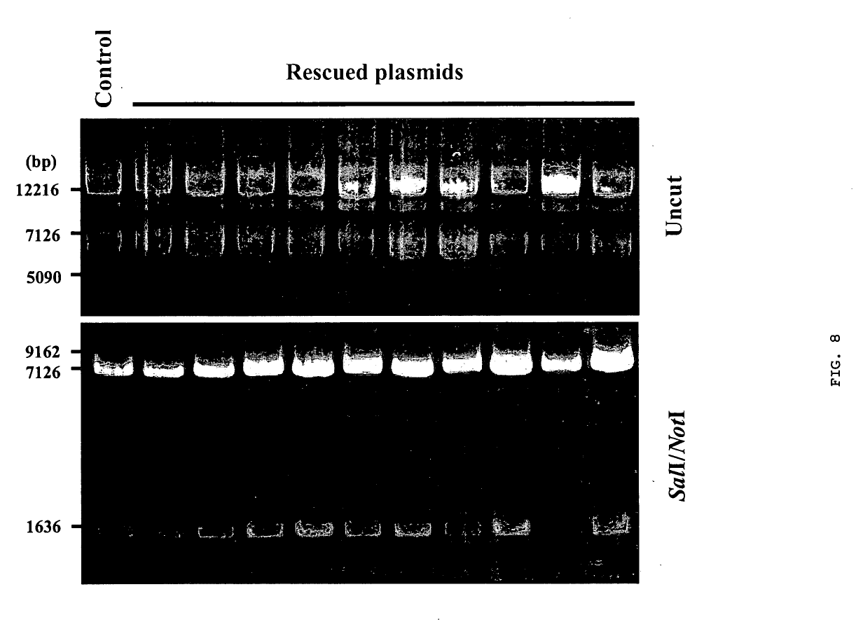

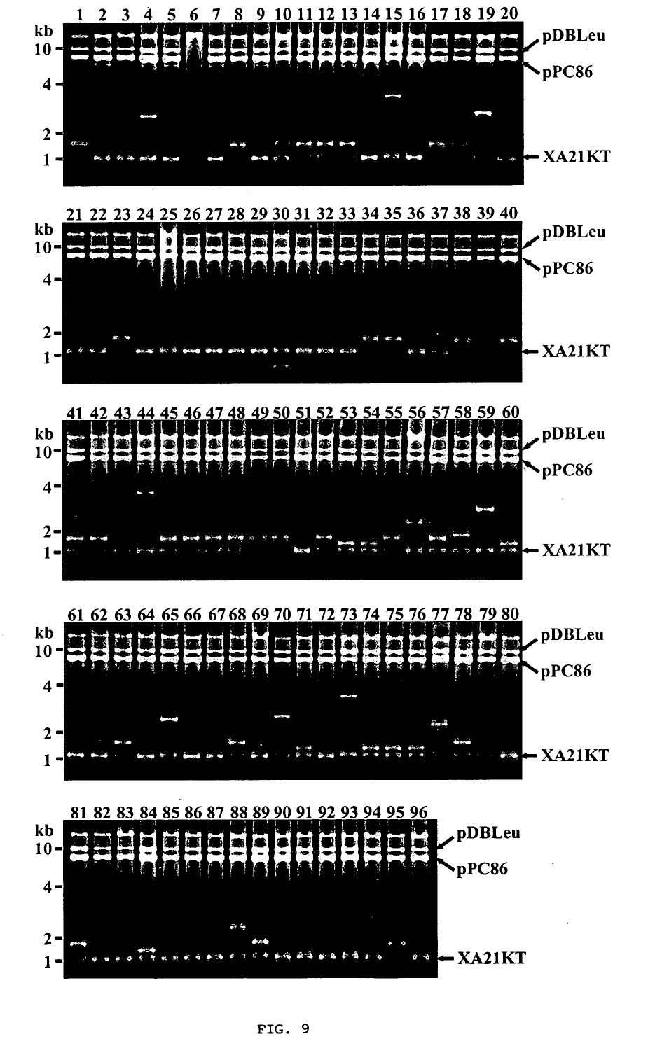

To confirm the prey identified from the above screening, the RCA products were transformed into the yeast strain MaV203 containing either pPC97-XA21KT or pPC97 empty vector. The transformants were first cultured in liquid selective medium (SD/-Leu-Trp) supplemented with cycloheximide (1 μg/mL) at 30° C. for 2 days without shaking and then grown on solid selective medium (SD/-Leu-Trp+cycloheximide) to identify colonies containing both bait and prey. These colonies were then spot-replicated onto selective medium (SD/-Leu-Trp-His+cycloheximide) to identify those carrying interacting proteins. FIG. 5 shows that most of the prey interact reproducibly with XA21KT, as indicated by the fast growth on the selective medium containing cycloheximide, but not with empty BD. One clone, indicated by the arrow, has grown comparably to the clones containing empty BD, indicating that the interaction defined by this clone is not reproducible.

The results therefore, indicate that the high-throughput RCA and cycloheximide counterselection is highly suitable for rapid analysis of candidate interactors. The entire bait and prey can be amplified from yeast lysates or isolated plasmids in a 96 well format. When retransformed into yeast, the original bait in the RCA products can be excluded by cycloheximide counterselection, which allows for the determination of specific interactions with other baits. To test reproducibility, the RCA products containing both bait and prey can be transformed into yeast. Since the experimental procedures for RCA and yeast retransformation are amenable to automation, this new strategy provides a basis to verify candidate interactors on a large scale. Such a verification would significantly reduce false positives, thereby increasing the quality of the interaction database created by the yeast two-hybrid approach.

### Example 5

### Isolation and Rescue Plasmid DNA from Single Colonies of Agrobacterium tumefaciens by Rolling Circle Amplification

Materials and Methods

The A. tumefaciens strain LBA4404 was obtained from Invitrogen (Carlsbad, Calif., USA) and the E. coli strain XL1-Blue was obtained from Stratagene (La Jolla, Calif., USA). The E. coli strain DY329 was a gift from Donald L. Court (National Cancer Institute, MD, USA). The TempliPhi™ 100 Amplification kit was purchased from Amersham Biosciences (Piscataway, N.J., USA) for RCA amplification. The plasmid pCmPU-GUS was constructed by inserting the beta-glucuronidase gene (GUS) into the binary vector pCmPU, a derivative of pCAMBIA1300 (Cambia, Canberra, Australia).

Protocol 1 RCA Amplification of Plasmid DNA from A. tumefaciens

1. Pick ⅓-½ of single bacterial colonies carrying pCmPU-GUS using toothpicks and suspend the cells into 5 μl of sample buffer.
2. Heat the cells at 95° C. for 3 minutes and then cool down to 4° C.
3. Add 5 μl of reaction buffer and 0.2 μl of Phi29 DNA polymerase to the cell suspensions.
4. Incubate at 30° C. for 6-18 hours for amplification and then incubate at 65° C. for 10 minutes to stop the reactions.
5. Transfer 3 μl of amplified DNA into a new tube and incubate with 10 units of the restriction enzyme SpeI in the appropriate buffer at 37° C. for 4 hours. Stop the reactions by heating the samples at 65° C. for 20 minutes.
6. One μl of RCA amplified DNA and 3 μl of SpeI digested RCA products are fractionated in 0.8% agarose gel.
   Protocol 2 Transformation of the E. coli Strain XL1-Blue with RCA Products
1. Digest 0.5 μl of RCA amplified DNA with a single-cut enzyme in 15 μl volumes. Stop the reactions by heating the samples at 65° C. for 20 minutes.
2. Self-ligation reactions are performed in 30 μl volumes containing 15 μl digested RCA products, 1×T4 DNA ligase buffer, and 400 units of T4 DNA ligase.
3. Incubate overnight at 16° C.
4. Mix 0.2 μl of self-ligated RCA products with 20 μl of XL1-Blue competent cells.
   Electroporate the cells using a Cell-Portator (GIBCO, BRL) according to manufacturer's instructions.
   Protocol 3 Transformation of the E. coli Strain DY329 with RCA Products
1. Inoculate 5 ml of LB medium containing 20 μg/ml of tetracycline with a fresh DY329 colony. Grow cells overnight at 32° C. with vigorous shaking (250 rpm).
2. Dilute 1 ml of overnight culture into 100 ml of LB medium containing 20 μg/ml of tetracycline. Incubate at 32° C. with vigorous shaking to an OD600=0.4-0.6.
3. Transfer cells to two 500-ml flasks. Heat shock one for 15 minutes in a 42° C. water bath with shaking (induced). Swirl the flask in an ice water slurry for 10 minutes to cool down. Cells in the other flask is kept on ice as a negative control (uninduced).
4. Centrifuge for 8 minutes at 5,500 g at 4° C. Discard the supernatant.
5. Resuspend the pellets in 1 ml of ice-cold sterile water and transfer the cells into two 1.5 ml Eppendorf tubes.
6. Centrifuge for 20 seconds at 4° C. at maximum speed. Discard the supernatant.
7. Repeat steps 5 to 6 twice. Resuspend the cells in 100 μl of ice-cold sterile water.
8. Mix 0.2 μl of RCA DNA with 20 μl of the induced and uninduced competent cells, respectively. Electroporate the cells using a Cell-Portator (GIBCO, BRL) according to manufacturer's instructions.

Due to low copy numbers of plasmid, verification of the constructs transformed into A. tumefaciens is usually achieved by Southern blot analysis. Alternatively, the isolated plasmid DNA from A. tumefaciens can be transformed into E. coli to propagate for subsequent studies. We directly amplified the entire plasmid pCmPU-GUS from single colonies of A. tumefaciens using RCA. The majority of these DNA products represent distinct concatemeric units of the pCmPU-GUS, which was confirmed by the restriction digestion. Some background could be generated from the genomic DNA of A. tumefaciens.

To recover the amplified plasmids, digestion of the RCA products with a single-cut restriction enzyme followed by self-ligation with T4 DNA ligase dramatically increased the bacterial transformation efficiency. For example, ˜5000 transformants were obtained from 50 ng of RCA amplified pCmPU-GUS (1.1×10⁵ cfu/μg DNA) using standard electroporation methods. Thus, RCA products can be efficiently transformed into E. coli after circularizing single units of linear concatemeric DNA.

To develop more generally applicable methods, we used the recombinogenic E. coli strain DY329. This strain was engineered with a λ prophage containing the recombination genes exo, bet, and gam under control of a temperature-sensitive λ cI-repressor (Yu et al. 2000). The bacterial cells become more recombinogenic when treated at 42° C. By using the transformation procedures described in the “Materials and methods”, an efficiency of 1.34×10⁴ cfu/μg DNA was achieved for the linear RCA DNA of pCmPU-GUS. In contrast, no transformants were obtained using similar amounts of RCA products when the DY329 cells were not treated by the 42° C. heat-shock, indicating that the engineered recombination activity is required for the transformation. Gel electrophoresis analysis indicated that the plasmids recovered from the transformants are similar in size to the monomeric pCmPU-GUS. Restriction digestion confirmed that these plasmids contained correct insert.

The RCA-based methods described herein, provide an alternative for the identification and recovery of transformed constructs from A. tumefaciens. Bacterial culturing and subsequent plasmid isolation using conventional methods are eliminated. No specific equipment is required. Amplification of entire plasmids for restriction digestion or even DNA sequencing allows detailed analysis of the constructs. The experimental steps are amenable to automation which is critical for processing samples in high-through manners.

Transforming DNA from other microorganisms into E. coli is a common strategy to purify, propagate, express and manipulate plasmid DNA. Our methods connect the powerful RCA to the well-established E. coli system. Plasmids could be amplified from other microorganisms or transformed cells, and introduced into E. coli for a variety of applications. For instance, by transforming RCA amplified plasmids from single yeast colonies into E. coli, we have been able to amplify and separate prey and bait constructs in two-hybrid analyses. The purified prey can then be used to test for protein-protein interactions with other constructs using similar assays. The E. coli transformation methods together with our previous yeast transformation procedures provide necessary the tools for RCA-based plasmid analyses and manipulation.

### REFERENCES

Blanco, L., Bemad, A., Lazaro, J. M., Martin, G., Garmendia, C. and Salas, M. 1989. Highly efficient DNA synthesis by the phage phi 29 DNA polymerase. Symmetrical mode of DNA replication. J. Biol. Chem. 264: 8935-8940.
Chern, M. S., Fitzgerald, H. A., Yadav, R. C., Canlas, P. E., Dong, X. and Ronald, P. C. (2001). Evidence for a disease-resistance pathway in rice similar to the NPR1-mediated signaling pathway in Arabidopsis. Plant J 27: 101-113.
Chevray, P. M. and Nathans, D. 1992. Protein interaction cloning in yeast: identification of mammalian proteins that react with the leucine zipper of Jun. Proc. Natl. Acad. Sci. USA 89: 5789-5793.
Dean, F. B., Nelson, J. R., Giesler, T. L. and Lasken, R. S. 2001. Rapid amplification of plasmid and phage DNA using phi29 DNA polymerase and multiply-primed rolling circle amplification. Genome Res. 11: 1095-1099.
Esteban, J. A., Salas, M., Blanco, L. 1993. Fidelity of phi 29 DNA polymerase. Comparison between protein-primed initiation and DNA polymerization. J. Biol. Chem. 268: 2719-2726.
Ewing, B. and Green, P. 1998. Base-calling of automated sequencer traces using phred. II. Error probabilities. Genome Res. 8:186-194.
Ewing, B., Hillier, L., Wendl, M C. and Green, P. 1998. Base-calling of automated sequencer traces using phred. I. Accuracy assessment. Genome Res. 8:175-185.
Fields, S. and Song, O. K. 1989. A novel genetic system to detect protein-protein interactions. Nature 340: 245-246.
Garmendia, C., Bernad, A., Esteban, J. A., Blanco, L. and Salas, M. 1992. The bacteriophage phi 29 DNA polymerase, a proofreading enzyme. J. Biol. Chem. 267: 2594-2599.
Hazbun, T. R. and Fields, S. 2001. Networking proteins in yeast. Proc Natl Acad Sci USA 98: 4277-4278.
Kornberg, A and Baker, T. A. 1992. DNA replication. W. H. Freeman and Company, San Francisco.
Soellick, T. R. and Uhrig J F. 2001. Development of an optimized interaction-mating protocol for large-scale yeast two-hybrid analyses. Genome Biol. 2: research0052.1-research0052.7.
Song, W. Y., Wang, G. L., Chen, L. L., Kim, H. S., Pi, L. Y., Holsten, T., Gardner, J., Wang, B., Zhai, W., X., Zhu, L. H., Fauquet, C. and Ronald, P. 1995. A receptor kinase-like protein encoded by the rice disease resistance gene, Xa21. Science 270:1804-1806.
Yin, Y. H., Zhu, Q., Dai, S. H., Lamb, C. and Beachy, R. N. 1997. RF2a, a bZIP transcriptional activator of the phloem-specific rice tungro bacilliform virus promoter, functions in vascular development. EMBO J. 16: 5247-5259.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All references mentioned herein, are incorporated by reference in their entirety.

1. A method for amplifying nucleic acid molecules from a cell, the method comprising the steps of:
providing an isolated cell;
administering a vector to the cell;
placing the cell in a reaction mixture comprising a DNA polymerase and at least one primer under reaction conditions that allow amplification of the nucleic acid molecule.

2. The method of claim 1, wherein the cell is a eukaryotic cell

3. The method of claim 1, wherein the cell is a yeast cell.

4. The method of claim 3, wherein the yeast cell is treated with an enzyme.

5. The method of claim 4, wherein the enzyme is zymolase.

6. The method of claim 1, wherein the DNA polymerase is selected from the group consisting of Φ29, Φ15, PZA, PZE, BS32, B103, Nf, M2, ΦPRD1, exo(−)VENT™, Klenow fragment of DNA polymerase I, T5 DNA polymerase, Sequenase, PRD1 DNA polymerase, and T4 DNA polymerase.

7. The method of claim 6, wherein the polymerase is a phage DNA polymerase.

8. The method of claim 6, wherein the phage DNA polymerase is Φ29 DNA polymerase.

9. The method of claim 1, wherein the nucleic acid molecule is amplified by rolling circle amplification.

10. The method of claim 1, wherein the nucleic acid molecule is circular DNA.

11. The method of claim 10, wherein the nucleic acid molecule is about 100 bases long up to 500,000 bases long.

12. The method of claim 1, wherein random primers hybridize to the nucleic acid molecule in the reaction mixture.

13. The method of claim 12, wherein the primers are identified by any one of SEQ ID NO's 1 through 8.

14. The method of claim 13, wherein one or any combination of primers identified by SEQ ID NO's 1 through 8 are administered to the reaction mixture.

15. The method of claim 13, wherein primers with at least about 45% homology to the primers identified by any one of SEQ ID NO's 1-8 amplify nucleic acid molecules.

16. The method of claim 13, wherein primers with at least about 50% homology to the primers identified by any one of SEQ ID NO's 1-8 amplify nucleic acid molecules.

17. The method of claim 13, wherein primers with at least about 75% homology to the primers identified by any one of SEQ ID NO's 1-8 amplify nucleic acid molecules.

18. The method of claim 13, wherein primers with at least about 80% homology to the primers identified by any one of SEQ ID NO's 1-8 amplify nucleic acid molecules.

19. The method of claim 13, wherein primers with at least about 95% homology to the primers identified by any one of SEQ ID NO's 1-8 amplify nucleic acid molecules.

20. The method of claim 12, wherein the primers comprise at least one modified base.

21. The method of claim 12, wherein the primers comprise about two modified bases.

22. The method of claim 12, wherein the primers comprise up to 8 modified bases.

23. The method of claim 1, wherein the vector comprises a selectable marker.

24. The method of claim 23, wherein the selectable marker is an auxotrophic or antibiotic resistance marker.

25. The method of claim 1, wherein the nucleic acid molecules are amplified with high fidelity with an error rate between about 10⁻⁶-10⁻¹⁰.

26. The method of claim 1, wherein the high fidelity amplified DNA is digested and sequenced without further purification.

27. The method of claim 26, wherein the high fidelity amplified DNA is transformed into yeast.

28. A high-throughput method for identifying candidate nucleic acid molecule interactors in a yeast two-hybrid screening comprising: method for amplifying DNA in a two-hybrid yeast cell comprising:
providing an isolated cell;
administering a bait vector to the cell;
administering a vector comprising prey nucleic acid sequences and a selection marker to the cell;
placing the cell in a reaction mixture comprising a DNA polymerase and at least one primer under reaction conditions that allow amplification of the nucleic acid molecules; and,
transforming cells with the amplified products and grown on a counter-selection medium;
thereby, identifying candidate interactors.

29. The method of claim 29, wherein the nucleic acid molecules are amplified by rolling circle amplification (RCA).

30. The method of claim 29, wherein the prey vector comprises a selection marker conferring resistance to transformed cells on a counterselection medium.

31. The method of claim 30, wherein the counterselection medium comprises cycloheximide.

32. The method of claim 31, wherein transformed cells comprising nucleic acid molecules with the selection marker are resistant to cycloheximide.

33. The method of claim 32, wherein transformed cells grown on the counterselection medium selectively grow as individual colonies lacking bait nucleic acid molecules.

34. The method of claim 32, wherein the transformed cells are yeast cells.

35. The method of claim 33, wherein nucleic acid molecules obtained from individual transformed cell colonies require no purification step.

36. The method of claim 29, wherein candidate interactor nucleic acid molecules are identified by sequence analysis.

37. A method for identifying a compound that interacts with amplified gene isolated from a mammal, comprising
contacting a candidate agent with the an amplified gene, an allele or fragment thereof, or expression product thereof; and
performing a detection step to detect interaction between the gene, an allele or fragment thereof, or expression product thereof.

38. The method of claim 36, wherein the candidate compound is selected from the group consisting of a protein, a peptide, an oligopeptide, a nucleic acid, a small organic molecule, a polysaccharide and a polynucleotide.

39. The method of claim 37 or 38 wherein the gene, variants or fragments thereof, or oligopeptides or candidate compound comprises a label.

40. The method of claim 36, wherein the gene is isolated from a mammal suffering from or susceptible to a disease.

41. The method of claim 40, wherein the disease is hereditary, a tumor, or caused by an infectious agent.

42. The method of claim 41, wherein the infectious agent is a virus, bacterium, protozoan or fungus.

43. The method of claim 37, wherein the amplified gene, variant or fragment oligopeptide are provided on a solid support.

44. The method of claim 43, wherein binding of the candidate compound with the amplified gene, variant or fragment or oligopeptide is detected.

45. The method of claim 43, wherein the amplified gene, variant or fragment or peptides or candidate compound comprises a detectable label.

46. A drug compound obtained by a method of any one of claims 37 through 45.

47. A kit comprising
an isolated yeast cell;
zymolase;
a vector;
a reaction mixture comprising a DNA polymerase;
primers identified by any one of SEQ ID NO's 1-8.

48. The kit of claim 47, wherein instructions for carrying out the method are provided.

49. A method for identifying a component of a test sample, comprising:
contacting a test sample with an amplified gene, variant or fragment thereof, or expression product of the amplified gene, variant or fragment thereof; and detecting interaction of the test sample with the amplified gene, an variant or fragment thereof, or expression product of the amplified gene, variant or fragment thereof.

50. The method of claim 49, wherein the test sample is a mammalian tissue or fluid sample.

51. A method for identifying one or more genes that mediate susceptibility to disease susceptibility in a mammal comprising:
amplifying nucleic acid molecules from a mammal, hybridizing an isolated nucleic acid sequence with a nucleic acid probe to form a hybridized molecule; and
detecting sequences hybridized to the probe.

52. The method of claim 51, wherein the amplified gene, allele or fragment oligopeptide are provided on a solid support.

53. The method of claim 52, wherein binding of the candidate gene and/or gene product with the amplified gene, allele or fragment or oligopeptide is detected.

54. The method of claim 51, wherein the amplified gene sequence is compared known genes in a database.

55. The method of claim 54, wherein the amplified gene is identified from the database.

56. The method of claim 55, wherein the database is GenBank, Human genome project or EMBL.

57. A composition for rolling circle amplification of nucleic acid molecules comprising:
an isolated yeast cell;
zymolase;
a vector with a selectable marker;
a reaction mixture comprising a DNA polymerase; and,
primers identified by any one of SEQ ID NO's 1-8.


SEQUENCE LISTING (8 sequences)

SEQ ID NO: 1 - DNA, 32 bp, Artificial
  Universal Primer
aggtcgaccccgggaatgaaaggccacccatt

SEQ ID NO: 2 - DNA, 40 bp, Artificial
  Universal Primer
ggggtaccggatcctcaaaattcaaggctcccaccttcaa

SEQ ID NO: 3 - DNA, 17 bp, Artificial
  Universal Primer
tcatcggaagagagtag

SEQ ID NO: 4 - DNA, 21 bp, Artificial
  Universal Primer
agggatgtttaataccactac

SEQ ID NO: 5 - DNA, 18 bp, Artificial
  Universal Primer
ttgattggagacttgacc

SEQ ID NO: 6 - DNA, 21 bp, Artificial
  Universal Primer
cccaaccaagaagtatgctga

SEQ ID NO: 7 - DNA, 23 bp, Artificial
  Universal Primer
tcacagggaaggagacaatggca

SEQ ID NO: 8 - DNA, 24 bp, Artificial
  Universal Primer
atgaaatcatgtgttgttgtggca